(19) United States
(12) Patent Application Publication
(10) Pub. No.: US 2020/0080061 A1
(43) Pub. Date: Mar. 12, 2020

(54) COMPOSITIONS AND METHODS FOR BIOENGINEERED TISSUES

(71) Applicant: The University of North Carolina at Chapel Hill, Chapel Hill, NC (US)

(72) Inventors: Arial Dawn Hanson, Somerville, MA (US); Mitsuo Yamauchi, Chapel Hill, NC (US); Eliane Lucie Wauthier, Chapel Hill, NC (US); Timothy Anh-Hieu Dinh, Chapel Hill, NC (US); Praveen Sethupathy, Hillsborough, NC (US); Lola M. Reid, Chapel Hill, NC (US)

(73) Assignee: The University of North Carolina at Chapel Hill, Chapel Hill, NC (US)

(21) Appl. No.: 16/681,660

(22) Filed: Nov. 12, 2019

Related U.S. Application Data
(62) Division of application No. 15/586,061, filed on May 3, 2017, now abandoned.
(60) Provisional application No. 62/335,013, filed on May 11, 2016.

(51) Int. Cl. C12N 5/071; A61L 27/36; A61L 27/38; C12N 5/00; A61L 27/56; A61L 27/26; A61L 27/24

(52) U.S. Cl. CPC C12N 5/0671; A61L 2430/28; C12N 5/067; A61L 27/3813; C12N 5/0068; A61L 27/56; A61L 27/3895; A61L 27/3886; A61L 27/3834; A61L 27/3633; A61L 27/3625; A61L 27/26; A61L 27/24; C12N 2533/90; C12N 2501/20; C12N 2501/998; C12N 2533/54; C12N 2535/00; C12N 2500/36; C12N 2500/34; C12N 2500/32; C12N 2500/25; C12N 2500/24; C12N 2500/14; C12N 2500/02; A61L 27/3691

(57) ABSTRACT

The present disclosure provides methods for producing bioengineered tissue along with an apparatus and other relevant compositions employed in generation thereof.

### CROSS REFERENCE TO RELATED APPLICATIONS

This application is a divisional of U.S. patent application Ser. No. 15/586,061, filed May 3, 2017, which claims priority to U.S. Provisional Patent Application No. 62/335,013, filed May 11, 2016, the entirety of which are incorporated herein by reference in their entirety.

### BACKGROUND

The following discussion of the background of the invention is merely provided to aid the reader in the understanding the invention and is not admitted to describe or constitute prior art to the present invention.

Spheroid and organoid culture systems and other organ modeling methods facilitate the formation of cell configurations and polarities that are closer to those found in native tissue. While cultures derived entirely from cloned cell populations have certain advantages, there is increasing recognition in regenerative medicine of the importance of three-dimensional organization, cell polarity, epithelial-mesenchymal interactions and the paracrine signals from the epithelial-mesenchymal relationships that serve to stabilize the cells and their functions.

The prior methods of growing organs and organoid tissues are constrained to mini-scale models since large numbers of cells cannot be sustained in the absence of vascular support and tissues that do not mimic the vascular and histological zonation of the model organs. Thus, there remains a need for scalable, stable methods of generating bioengineered tissues.

### SUMMARY OF THE INVENTION

Aspects of the disclosure relate to compositions, kits, and methods for producing and using a bioengineered tissue or micro-organ and a container configured for the generation thereof.

Aspects of the disclosure relate to a container for the generation of bioengineered tissue. In some embodiments, the generation comprises introducing epithelial cells and/or mesenchymal cells into or onto a biomatrix scaffold. In some embodiments, the generation comprises introducing parenchymal and/or non-parenchymal cells. In some embodiments the cells are lineage stage partners of one another. Aspects of the disclosure relate to a three-dimensional scaffold comprising extracellular matrix, which in turn comprises (i) native collagens found in an organ and/or (ii) matrix remnants of a vascular tree found in an organ.

In some embodiments, the biomatrix scaffold comprises collagens. In some embodiments, the biomatrix scaffold comprises (1) (i) nascent collagens, (ii) aggregated but not cross-linked collagen molecules, (iii) cross-linked collagens and (iv) factors (matrix components, signaling molecules, other factors) bound to these different forms of collagens and/or (2) the vast majority of both cross-linked and uncross-linked native collagens found in the tissue along with matrix molecules and signaling molecules bound to these collagens. In some embodiments, the biomatrix scaffold is three dimensional. In some embodiments, the biomatrix scaffold comprises one or more collagen associated matrix components such as laminins, nidogen, elastins, proteoglycans, hyaluronans, non-sulfated glycosaminoglycans, and sulfated glycosaminoglycans and growth factors and cytokines associated with the matrix components. In some embodiments, the biomatrix scaffold comprises greater than 50% of matrix-bound signaling molecules found in vivo. In some embodiments, the matrix-bound signaling molecules may be epidermal growth factors (EGFs), fibroblast growth factors (FGFs), hepatocyte growth factors (HGFs), insulin-like growth factors (IGFs), transforming growth factors (TGFs), nerve growth factors (NGFs), neurotrophic factors, interleukins, leukemia inhibitory factors (LIFs), vascular endothelial cell growth factors (VEGFs), platelet-derived growth factors (PDGFs), stem cell factor (SCFs), colony stimulating factors (CSFs), GM-CSFs, erythropoietin, thrombopoietin, heparin binding growth factors, IGF binding proteins, placental growth factors, and wnt signals. In some embodiments, the biomatrix scaffold comprises a matrix remnant of the vascular tree of the tissue. In further embodiments, the matrix remnant may provide vascular support of the cells in the bioengineered tissue

In some embodiments, where the cells are in a seeding medium, the generation may optionally further comprise replacing the seeding medium with a differentiation medium after an initial incubation period. In some embodiments, where the cells are in a seeding medium, they are introduced in multiple intervals, each interval followed by a period of rest. In some embodiments, the interval is about 10 minutes and the period of rest is about 10 minutes. In some embodiments, the seeding density is less than or about 12 million cells per gram of wet weight of the biomatrix scaffolds and introduced in one or more intervals. In some embodiments, the cells in the seeding medium are introduced at a rate of ˜15 ml/min for one or more intervals. In some embodiments, the cells in the seeding medium are introduced in 10 minute intervals, each followed by a 10 minute period of rest. In some embodiments, the cells in the seeding media are introduced at a rate of 1.3 ml/min after three intervals.

In some embodiments, the seeding medium comprises a seeding medium that is serum-free. In some embodiments, the seeding medium is supplemented with serum, optionally between about 2% to 10% fetal serum such as fetal bovine serum (FBS). In some embodiments, serum supplementation of the medium may be necessary (e.g. to inactivate enzymes used in preparing cell suspension). In some embodiments, this supplementation occurs over a few hours.

In some embodiments, the seeding medium comprises basal medium, lipids, insulin, transferrin, and/or antioxidants. In some embodiments, the seeding medium may comprise one or more of the following: a basal medium, low calcium (0.3-0.5 mM), no copper, zinc and selenium, insulin, transferrin/fe, and one or more purified free fatty acids (e.g. palmitic acid, palmitoleic acid, stearic acid, oleic acid, linoleic acid, linolenic acid) optionally complexed with purified albumin, and one or more lipid-binding proteins such as high density lipoprotein (HDL). In some embodiments, the seeding medium may be used, comprises, or maintains low oxygen concentration levels (1-2%).

In some embodiments, the cells are incubated at 4° C. in the seeding medium for 4 to 6 hours prior to the introduction step. In some embodiments, the cells may be isolated from a fetal or neonatal organ. In some embodiments, the mesenchymal cells are stromal, endothelia, or hemopoietic cells. In some embodiments, the cells may be isolated from an adult or child donor. In some embodiments, the epithelial or parenchymal cells may be any one or more of biliary tree stem cells, gall bladder-derived stem cells, hepatic stem cells, hepatoblasts, committed hepatocytic and biliary progenitors, axin2+ progenitors (e.g. axin2+ hepatic progenitors), mature parenchymal or epithelial cells, mature hepatocytes, mature cholangiocytes, pancreatic stem cells, pancreatic committed progenitors, islet cells, and/or acinar cells and/or the mesenchymal or non-parenchymal cells may be any one of angioblasts, stellate cell precursors, stellate cells, mesenchymal stem cells, pericytes, smooth muscle cells, stromal cells, neuronal cell precursors, neuronal cells, endothelial cell precursors, endothelial cells, hematopoetic cell precursors, and/or hematopoetic cells. In some embodiments, the epithelial or parenchymal cells may be stem cells and/or descendants thereof from the biliary tree, liver, gall bladder, hepato-pancreatic common duct and/or the mesenchymal or non-parenchymal cells may be angioblasts, endothelial and/or stellate cell precursors, mesenchymal stem cells, stellate cells, stromal cells, smooth muscle cells, endothelia, bone marrow-derived stem cells, hematopoetic cell precursors, and/or hematopoetic cells. In some embodiments, the epithelial or parenchymal cells may include differentiated parenchymal cells, such as but not limited to axin2+ progenitors (e.g. axin2+ hepatocytes or hepatic progenitors), mature cells (e.g. mature hepatocytes, mature cholangiocytes), polyploid cells (e.g. polyploid hepatocytes) and apoptotic cells. In some embodiments, mature cells may be associated with sinusoidal endothelia, some of which may be fenestrated mesenchymal cells (e.g. endothelial cells). In some embodiments; the axin2+ progenitor cells (e.g. axin2+ hepatic progenitors) may be tethered to endothelial cells. In some embodiments, the epithelial or parenchymal cells are mature islets, optionally associated with mature endothelia, and/or mature acinar cells, and/or optionally associated with mature stroma. In some embodiments, the ratio of cells is 80% to 20%—epithelial to mesenchymal or parenchymal to non-parenchymal. In some embodiments, the cells are at least 50% stem cells and/or precursor cells. In some embodiments, the cells do not comprise any terminally differentiated hepatocytes and/or pancreatic cells. In some embodiments, the epithelial or parenchymal cells may be one or more of stem cells, committed progenitors, diploid adult cells, polyploid adult cells, and/or terminally differentiated cells and/or the mesenchymal or non-parenchymal cells may be one or more of angioblasts, precursors to endothelia, mature endothelia, precursors to stroma, mature stroma, neuronal precursors and mature neuronal cells, precursors to hemopoietic cells, and/or mature hemopoietic cells.

In some embodiments, the composition of the cells may be adjusted for the desired tissue, e.g. hepatic cells may be used in specific proportions for bioengineered liver tissue or pancreatic cells may be used in specific proportions for bioengineered pancreatic tissues. For example, for liver, epithelial cells may be one or more of stem cells (e.g. biliary tree stem cells) and their descendants from the biliary tree, liver, hepato-pancreatic common duct, and/or gall bladder, biliary tree stem cells, gallbladder-derived stem cells, hepatic stem cells, hepatoblasts, committed hepatocytic and biliary progenitors, axin2+ progenitors (e.g. axin2+ hepatic progenitors), mature hepatocytes, and/or mature cholangiocytes; and/or the mesenchymal or non-parenchymal cells may be one or more of angioblasts, stellate cell precursors, stellate cells, mesenchymal stem cells, smooth muscle cells, stromal cells, endothelial cell precursors, endothelial cells, hematopoetic cell precursors, and/or hematopoetic cells. Similarly, these same mesenchymal or non-parenchymal cells may be used for pancreas; and/or epithelial cells for the pancreas may include biliary tree stem cells (e.g. those from the hepato-pancreatic common duct), pancreatic stem cells, pancreatic committed progenitors, islet cells, stem cells and their descendants from the biliary tree, hepato-pancreatic common duct, or pancreas and/or acinar cells. In further embodiments, for liver, terminally differentiated hepatocytes may be excluded and, for pancreas, terminally differentiated pancreatic cells may be excluded.

In some embodiments, where a differentiation medium is used, the differentiation medium comprises basal medium, lipids, insulin, transferrin, antioxidants, copper, calcium, and/or one or more signals for the propagation and/or maintenance of one or more of the epithelial cells, mesenchymal cells, parenchymal cells, and/or non-parenchymal cells—depending on the cells used. Aspects of the disclosure relate to the differentiation medium itself. In some embodiments, the differentiation medium may include Kubota's Medium; one or more lipid binding proteins (e.g. HDL), one or more purified fatty acids (e.g. palmitic acid, palmitoleic acid, stearic acid, oleic acid, linoleic acid, linolenic acid), one or more sugars (galactose, glucose, fructose), one or more glucocorticoids (e.g. dexamethasone or hydrocortisone), copper (e.g. at a concentration of approximately or about 10⁻¹⁰ to approximately or about 10⁻¹² M); calcium (e.g. at a concentration of 0.6 mM); one or more hormones and/or growth factors for the propagation and/or maintenance of epithelial or parenchymal cells selected from prolactin, growth hormone, glucocorticoids, glucagon, thyroid hormones (e.g. tri-iodothryronine or T3), epidermal growth factors (EGFs), hepatocyte growth factors (HGFs), fibroblast growth factors (FGFs), insulin-like growth factors (IGFs), leukemia inhibitor factor (LIF), interleukins (IL) such as IL6 and IL11, wnt ligands, bone morphogenetic proteins (BMPs), and/or cyclic adenosine monophosphate, and/or one or more hormones and/or growth factors for the propagation and/or maintenance of mesenchymal or non-parenchymal cells selected from angiopoietin, vascular endothelial cell growth factors (VEGFs), interleukins (ILs), stem cell factors (SCFs), leukemia inhibitory factor (LIF), colony stimulating factors (CSFs), thrombopoietin, platelet derived growth factors (PDGFs), erythropoietin, insulin-like growth factors (IGFs), fibroblast growth factors (FGFs), epidermal growth factors (EGFs). In some embodiments, the differentiation medium may be used, comprises or maintains oxygen levels at approximately 5%.

In some embodiments, the container is designed for a flow path for fluids that is designed to mimic vascular support of cells.

Aspects of the disclosure relate to bioengineered tissue comprising zonation-dependent phenotypic traits characteristic of native liver, said phenotypic traits including (a) periportal region having traits of stem/progenitor cells, diploid adult cells, and/or associated mesenchymal or non-parenchymal precursor cells, (b) a mid-acinar region having cells with traits of mature biliary epithelia (e.g. cholangiocytes) and/or associated mature stellate and stromal cells, sinusoidal plates of mature parenchymal cells (e.g. hepatocytes) and/or associated mesenchymal cells, such as but not limited to the sinusoidal endothelia and/or pericytes (i.e. smooth muscle cells), (c) a pericentral region having traits of terminally differentiated parenchymal cells, such as but not limited to hepatocytes, including polyploid hepatocytes and apoptotic hepatocytes, and/or associated mesenchymal cells, such as but not limited to fenestrated endothelia and/or diploid axin2+ hepatic progenitors tethered to endothelia. In some embodiments, the phenotypic traits of the tissue include traits associated with diploid parenchymal and/or mesenchymal cells of the periportal zone. In some embodiments, the phenotypic traits of the tissue include traits of mature parenchymal (e.g. mature hepatic parenchymal cells) and/or mesenchymal cells (e.g. sinusoidal endothelia) found in the mid-acinar region of native liver. In some embodiments, the phenotypic traits of the tissue include traits of parenchymal (e.g. hepatic parenchymal cells) and/or mesenchymal cells of the pericentral zone. In some embodiments, the tissue comprises one or more of (i) polyploid hepatocytes associated with fenestrated endothelial cells, and/or (ii) diploid hepatic progenitors periportally and/or axin2+ hepatic progenitors connecting to endothelia of a central vein. In some embodiments, the periportal region of the tissue is enriched in traits of the stem/progenitor cell niches that comprise hepatic stem cells, hepatoblasts and/or committed progenitors and/or diploid adult hepatocytes. In some embodiments, the parenchymal cells of the tissue further comprise precursors and/or mature forms of hepatocytes and/or cholangiocytes. In some embodiments, the mesenchymal cells of the tissue further comprise precursors and/or mature forms of stellate cells, pericytes, smooth muscle cells and/or endothelia. Similar, aspects of the disclosure relate to a bioengineered tissue comprising zonation-dependent phenotypic traits characteristic of native pancreas and/or that includes zonation associated with pancreatic cells in the head of the pancreas and those associated with pancreatic cells in the tail of the pancreas. In some embodiments, the mesenchymal cells include stroma, smooth muscle cells, endothelia and hematopoietic cells; in further embodiments, these mesenchymal cells may be indicative of zonation dependent traits.

Further aspects relate to a three-dimensional micro-organ. Non-limiting examples include a three-dimensional micro-organ generated in the disclosed container or comprised of the disclosed bioengineered tissue. Kits for the generation and culture of these micro-organs are also contemplated herein.

Also provided herein is a method of evaluating a treatment for an organ comprising administering the treatment to a bioengineered tissue or a three-dimensional micro-organ.

### BRIEF DESCRIPTION OF THE DRAWINGS

FIGS. 1A-1P shows the characterization of biomatrix scaffold following decellularization. A) The percentage of retention of diverse growth factors in the biomatrix scaffold compared to that in fresh tissue. B-E) Ultrastructure of biomatrix scaffold imaged by scanning electron microscopy (SEM). B) Portal triad containing the portal vein (PV), hepatic artery (HA) and bile ducts (arrows). C) The sinusoidal region of the acinus in the biomatrix scaffolds indicating that it is void of cells D-E) Collagen bundles (*) and adhesion molecules bound to the collagens (arrows). F-O) Immunohistochemistry identifying matrix molecules in their proper zonal locations within the liver acinus P) Quantitative analysis of collagen content in the scaffolds compared to that in fresh tissue.

FIG. 2 depicts RNA sequencing data of relative gene expression between cells obtained from the three fetal liver tissues and used in the bioreactors

FIGS. 3A-3I shows histology of human fetal liver stem/progenitor cells following 14 days in culture. A-F) Markers of cells located in the periportal region. G) Periodic acid shift (PAS) staining of hepatic cells demonstrating glycogen storage. H) Hepatocytes positive for Cyp3A4, a P450 metabolism enzyme. I) SEM image of endothelial cells lining a vessel. The inserted image is of endothelial cells positive for CD31, also called platelet endothelial cell adhesion molecule (PECAM).

FIGS. 4A-4E depicts RNA-sequencing relative expression of fetal liver, bioreactor tissue (Bio_T14), and adult liver samples. A) Matrix metallopeptidases (MMP) such as MMP-2 and -9, are enzymes involved in matrix remodeling. B-E) Expression of extracellular matrix molecules. The cells grown in the bioreactors express significantly higher levels of ECM molecules compared to the other samples (p<0.05). [Bio_T14=bioreactor number T14)

FIG. 5 depicts RNA-sequencing relative gene expression of markers that profile cells found in the periportal region. Cells cultured in the bioreactor had a significant decrease in gene expression of stem cell and hepatoblast markers, and an increase in cholangiocyte markers p<0.05. This suggests a shift towards a more mature phenotype. p<0.05

FIG. 6 depicts RNA-sequencing relative gene expression of markers that profile cells found in the pericentral region. In parallel to a decrease in stem cell and progenitor cell markers, cells cultured in the bioreactor continued to differentiate towards a mature hepatic phenotype, evident by the increased expression of genes associated with mature metabolic traits. p<0.05

FIGS. 7A-7C shows the results of expression assays A) RNA sequencing expression of genes related to the feedback loop and signal transduction pathway called the Salvador/Warts/Hippo (SWH) pathway that regulates organ size and involving Hippo (“hippopotamus-like”) kinases and YAP (Yes associated protein) Cells cultured in the bioreactor show a decrease in Hippo kinase and a rise in YAP and associated targeting genes, compared to fetal and adult liver, suggesting an ongoing regenerative process. B) Gene expression of angiogenic markers and SEM image of fetal liver endothelial cells lining a vessel in the biomatrix scaffold after 14 days in culture. C) Relative gene expression of hematopoietic and endothelial stem cell markers such as the endothelial transcription factor, GATA-2, stem cell factor receptor (SCR) and interleukin 7R (IL7R) and mature hematopoietic genes such as recombinant activating gene 1 (Rag1), CD3 (T-cell co-receptor) and colony stimulating factor (CSF). Bioreactor samples have gene expression levels of CD3 similar to that found in adult liver and with rising Rag1 expression, both associated with T cells. CSF, a gene expressed by myeloid cells, is significantly higher compared to that in both fetal and adult livers. p<0.05

FIGS. 8A-8B shows the results of various assays: A) Cell viability indicated by lactate dehydrogeniase (LDH), full length keratin 18 (FL-K18), an indicator of necrosis, and cleaved cytokeratin 18 (ccK18) an indicator of apoptosis; and B) cell production of alpha-fetoprotein (AFP) and albumin and secretion of urea over 14 days in culture. The rise and fall in albumin levels seemed to complement the apoptosis data, suggestive of a cell cycle phenomenon and a regenerative response.

FIGS. 9A-9C show cells cultured in the bioreactors and undergoing either gluconeogenesis or glycolysis. The shift in either production or consumption of glucose may also correspond to a shift in development of the tissue-engineered liver. Gluconeogenesis occurs in precursor and periportal cells, whereas glycolysis is associated with cells in the pericentral region. B) Multivariable analysis indicating that the metabolic behavior of the bioreactors, while trending similarly, are still at different stages of metabolic function. C) The variable importance in projection (VIP) plot shows the metabolites that contribute to the separation. VIP>1.0 is considered important.

FIGS. 10A-10F are transmission electron microscopy (TEM) images of cells in the tissue-engineered liver following 14 days in culture. A-C) Several hepatocyte-like cells forming bile canaliculi (BC) and sinusoidal spaces between them (arrow). B) Possible secretory vesicles are seen around the bile canaliculi (arrow). D) Cells adherent to biomatrix scaffold. E, F) Junctional complexes between cells including desmosomes, adherins and gap junctions (arrows).

FIG. 11 is an image of the decellularization process in a rat liver and yielding biomatrix scaffolds used in the bioreactor experiments.

FIG. 12 depicts albumin and urea secretion by hepatocytes when cultured in serum-free, hormonally-defined culture medium (BIO-LIV-HDM) designed for the bioreactors or commercially available hepatocyte maintenance medium (HMM).

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. For instance, descriptors may be used to refer to biological material (e.g. tissue, organoids, samples) exhibiting characteristics of a particular organ, e.g. the use of “hepatic” to describe liver-derived tissue or a liver-like organoid. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

### I. DEFINITIONS

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration (e.g., the percentage of collagen in the total proteins in the biomatrix scaffold) and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

The term bioengineered is used herein to describe a man-made organ or tissue engineered to have biological properties similar or identical to a naturally occurring organ or tissue. In some aspects, this may require the use of engineering of a particular apparatus; in other aspects, this may require the use of a variety of biological factors.

The term “biomatrix scaffold” refers to an isolated tissue extract enriched in extracellular matrix, and as described herein retains some, optionally many or most, of the collagens and/or collagen-bound factors found naturally in the biological tissue. In some embodiments the biomatrix scaffold comprises, consists of, or consists essentially of collagens, fibronectins, laminins, nidogen/entactins, integrins, elastin, proteoglycans, glycosaminoglycans (sulfated and non-sulfated—including hyaluronans) and any combination thereof, all being part of the biomatrix scaffold (e.g., encompassed in the term biomatrix scaffold).

In some embodiments, the biomatrix scaffold lacks a detectable amount of a specific collagen, fibronectin, laminins, nidogen/entactins, elastins, proteogylcans, glycosaminoglycans and/or any combination thereof. In some embodiments essentially all of the collagens and collagen-bound factors are retained and in other embodiments the biomatrix scaffold comprises all of the collagens known to be in the tissue.

The biomatrix scaffold may comprise at least about 50%, 60%, 70%, 75%, 80%, 85%, 90%, 95%, 97%, 98%, 99%, 99.5% or 100% of the collagens, collagen-associated matrix components, and/or matrix bound growth factors, hormones and/or cytokines, in any combination, found in the natural biological tissue. In some embodiments the biomatrix scaffold comprises at least 95% of the collagens and most of the collagen-associated matrix components and matrix bound growth factors, hormones and/or cytokines of the biological tissue. The collagens described herein may be nascent (newly formed), non-cross-linked collagens. As disclosed herein, collagens consist of 3 amino acid chains woven like hair into a triple helix (regions dominated by 3 amino acids: [glycine-proline-X] (where X can be any of a number of different amino acids), forming the fiber-like domain of the collagen and with ends of the molecule that have an amino acid chemistry that is unique to different collagen types and resulting in globular domains. The collagen molecules may be secreted; self-assemble to form collagen fibrils (aggregated collagen molecules); self-assemble with non-collagenous matrix components and with signaling molecules (cytokines, growth factors); and then are cross-linked to form the extracellular matrix. Exemplary collagens and methods of extraction thereof are described in brief herein below.

Certain collagen molecules have an amino acid chemistry that is unique to each of the 29 known collagen types. The collagens are secreted from cells and then one or both ends of the molecules are removed by specific peptidases followed by aggregation of multiple collagen molecules to form collagen fibers or fibrils. The exceptions are the “network collagens” that retain the globular domains and then aggregate end-on-end to form networks of collagen molecules (i.e. with chicken-wire-like structures). After aggregation into fibers or into networks, the collagens are cross-linked through the effects of lysyl oxidase, an extracellular copper-dependent enzyme that yields covalent bonding between collagen molecules (and also between elastin molecules) to produce cross-linked forms constituting very stable aggregates of collagens and anything bound to the collagens. The number of collagen molecules per fibril in the fibrillar collagens and the patterns of connections in the network collagens are dictated by the exact amino acid chemistry of the specific collagen type.

Extraction of a tissue to isolate uncross-linked as well as cross-linked collagens in an insoluble state may be accomplished utilizing buffers that are at neutral pH and with salt concentrations at or above 1 M; the exact concentration of the salt required to preserve the uncross-linked collagens as insoluble depends on the collagen types. For example, Type I and III collagens, found in abundance in skin, require approximately 1 M salt; by contrast the collagens in amniotic membranes (e.g. type V collagens) require 3.5-4.5 M salt); the uncross-linked as well as cross-linked collagens in liver require at least 3.4 M salt. Consequently, most methods of preparing extracts enriched in extracellular matrix do not preserve all of the collagens, especially those that are not crosslinked. In addition some methods make use of either a) enzymes that degrade matrix components and/or b) low salt or no salt buffers (e.g. distilled water) that result in dissolution of the uncross-linked collagens and any factors bound to them. Therefore, there are multiple forms of extracts for matrix scaffolds that contain cross-linked collagens and any factors bound to those cross-linked collagens but are devoid of or have minimal amounts of the uncross-linked collagens and their associated factors. Although the extracts that isolate primarily or solely the cross-linked collagens also have adhesion molecules and signaling molecules, these are not readily available to interact with the cells because of their orientation and location within the cross-linked matrix. By contrast, the uncross-linked collagens have self-assembled with other matrix components and with signaling molecules all of which are available for interactions with cells. In some embodiments, the biomatrix scaffold disclosed herein is prepared avoiding low ionic strength buffers to preserve both the cross-linked and non-cross-linked collagens.

In some embodiments, the biomatrix scaffold disclosed herein contain essentially all of the collagens comprising the nascent (newly formed) collagens, the aggregated collagen molecules prior to cross-linking, plus the cross-linked collagens. In addition, the biomatrix scaffold may optionally comprise other matrix components plus signaling molecules that are bound to these collagens or to bound matrix components. In some embodiments, the ratio of collagens in the biomatrix scaffold is similar or identical to the ratio in the tissue from which the biomatrix scaffold is derived. Non-limiting examples of a suitable percentage of nascent collagens to mimic the original tissue include, but are not limited to, at least about or about 0.05%, 0.1%, 0.5%, 1%, 5%, 10%, 15%, 20%, 25%, 30%, 35%, 40%, 45%, or 50%.

As described herein, “most of the collagen-associated matrix components and matrix bound growth factors, hormones and/or cytokines of the biological tissue” refers to the biomatrix scaffold retaining about 50%, 60%, 70%, 75%, 80%, 85%, 90%, 95%, 97%, 98%, 99%, 99.5% or 100% of the collagen-associated matrix components and matrix bound growth factors, hormones and/or cytokines found in the natural (e.g., unprocessed) biological tissue. The terms “powdered” or “pulverized” are used interchangeably herein to describe a biomatrix scaffold that has been ground into a powder. The term “three-dimensional biomatrix scaffold” refers to a decellularized scaffold that retains its native three dimensional structure. Such three-dimensional scaffold may be either whole scaffold or frozen sections thereof.

The terms “buffer” and/or “rinse media” are used herein to refer to the reagents used in the preparation of the biomatrix scaffold.

As used herein, the term “cell” refers to a eukaryotic cell. In some embodiments, this cell is of animal origin and can be a stem cell or a somatic cell. The term “population of cells” refers to a group of one or more cells of the same or different cell type with the same or different origin. In some embodiments, this population of cells may be derived from a cell line; in some embodiments, this population of cells may be derived from a sample of organ or tissue.

The term “progenitor cell” or “precursor” as used herein, is broadly defined to encompass both stem cells and their progeny; in some aspects of the disclosure, the term “stem/progenitor” will be used herein interchangeably with “progenitor,” “progenitor cell,” or “precursor” herein. “Progeny” may include multipotent stem cells or unipotent committed cells that can differentiate into a particular lineage leading to one or more mature cell types. Non-limiting examples of progenitor cells include but are not limited to embryonic stem (ES) cells, induced pluripotent stem (iPS) cells, germ layer stem cells, determined stem cells, perinatal stem cells, amniotic fluid-derived stem cells, mesenchymal stem cells, transit amplifying cells, or committed progenitor cells of any tissue type. When used with descriptors such as “unipotent,” “multipotent,” and/or “committed,”, the ability of the cells to differentiate to one or more adult fates is indicated—e.g. embryonic stem cells are pluripotent and capable of giving rise to all adult fates of the 3 germ layers (ectoderm, mesoderm, endoderm); the determined stem cells are multipotent and able to give rise to 2 or more adult fates; while stellate cell precursors or endothelial progenitor cells are examples of unipotent progenitors and so committed to a specific cell lineage.

As used herein, “parenchymal cells” are epithelial cells, typically of organs. In the liver, they may comprise hepatocytes and cholangiocytes; in the pancreas, they may comprise acinar cells and islets; in liver and pancreas and other endodermal organs (e.g. thyroid, intestine, lung), they may be derived from endodermal stem cells. Their phenotypic traits are lineage dependent with the earliest sets of traits found in cells in zone 1 of the liver acinus, transitioning to those in the mid-acinar zone (zone 2 of the liver), and ending in terminally differentiated cells in the pericentral zone (zone 3 of the liver). In addition, a population of diploid parenchymal cells linked to the endothelia forming the central vein has been newly discovered to have unipotent progenitor properties. Non-limiting exemplary parenchymal cells are biliary tree stem cells, hepatic stem cells, hepatoblasts, committed hepatocytic and biliary progenitors, axin2+ progenitors (e.g. axin2+ hepatic progenitors), mature parenchymal cells (hepatocytes, cholangiocytes, and multipotent or unipotent derivatives of the stem cell subpopulations thereof). Further non-limiting examples include, biliary tree stem cells, especially from the hepato-pancreatic common duct, pancreatic stem cells, pancreatic committed progenitors from the hepato-pancreatic common duct and from pancreatic duct glands, islets and acinar cells. These exemplary embodiments may be useful in, for example, the liver and pancreas, respectively.

As used herein, “non-parenchymal cells” are those derived from mesodermal and ectodermal stem cells and their lineage descendants including mature mesodermal and ectodermal cell types. The mesodermal stem cell-derived progeny include angioblasts, populations of precursors to endothelia and stellate cells, mature endothelia, mature stellate cells, stromal cells, smooth muscle cells, pericytes, hematopoietic stem cells and progenitors and their descendants that include Kupffer cells, natural killer cells (Pit cells), myeloid cells, lymphocytes, and various other hemopoietic cells. The ectodermal stem cell progeny include neuronal precursors and mature neuronal cells.

“Epithelial cells” are known in the art to be those derived from epithelium. As used herein, the term “mesenchymal cell” refers to those non-parenchymal cells that are mesodermal in origin. There is an epithelial-mesenchymal partnership constituting a relational centerpiece of a tissue, and it may be lineage dependent; that is the epithelial stem cells are partnered with a mesenchymal stem cell and their descendants mature in a coordinate fashion. The relationship is sustained by “cross-talk” of signals (paracrine signals) comprised of soluble signals and extracellular matrix components that work dynamically and synergistically to regulate biological responses of the epithelia and of the mesenchymal cells. For example, angioblasts (a type of mesenchymal stem cell population) are partnered with the hepatic stem cells. They give rise to endothelial cell precursors and their descendants that are partnered with the hepatocytic lineage, and, in parallel, to stellate cell precursors and their descendants that are partnered with the cholangiocytic lineage. The stellate and endothelial cell populations undergo a maturational process that parallels that of and is coordinate with the epithelial cells to which they are bound. Thus, the phenotypic properties of these cells are lineage dependent and are distinct depending on whether the cells are at early, intermediate or late stages of the lineage. This translates roughly to whether the cells are from zone 1 (early), zone 2 (intermediate), or zone 3 (late) of the liver acinus. Non-limiting exemplary non-parenchymal cells are angioblasts, mesenchymal stem cells, stellate cell precursors, stellate cells, pericytes, stromal cells, smooth muscle cells, neuronal cell precursors, neuronal cells, endothelial cell precursors, endothelial cells, hematopoetic cell precursors, and hematopoetic cells.

The term “biliary tree stem cells” (BTSCs) refers to stem cells found throughout the biliary tree, including in the gall bladder, with the ability to transition into hepatic and/or pancreatic stem cells and their descendant progenitor cells. They are found in both the extramural peribiliary glands (PBGs)—tethered to the surface of the bile ducts—and the intramural PBGs—within the bile duct walls. Descendants of the PBG-associated BTSCs are found in the gallbladder and located at the or the bottoms of the gallbladder villi, in niches that have parallels with intestinal crypts. There are multiple BTSC subpopulations and that form a lineage that transition to hepatic stem cells (HpSCs) found in the PBGs of the large intrahepatic bile ducts and that connect into the ductal plates (fetal and neonatal tissue) and that convert to canals of Hering (pediatric and adult tissue). The HpSCs give rise to hepatoblasts, located adjacent to or near to the canals of Hering and transition into committed hepatocytic and cholangiocytic progenitors that mature into hepatocytes and cholangiocytes. In addition, there are descendants of the BTSCs that give rise to pancreatic stem cells found throughout the biliary tree but primarily within the PBGs of the hepato-pancreatic common duct, and; these, in turn, transition to committed pancreatic progenitors found in the pancreatic duct glands within the pancreas. The biomarkers for all of the BTSC subpopulations include endodermal transcription factors (SOX9, SOX17, FOXL1, HNF4-alpha, ONECUT2, PDX1), pluripotency genes (e.g. OCT4, SOX2, NANOG, SALL4, KLF4, KLF5, BMI-1); one or more of the isoforms of CD44, (both CD44s and CD44v), the hyaluronan receptors isoforms; CXCR4; ITGB1 (CD29), ITGA6 (CD49f), ITGB4, and cytokeratins 8 and 18. The isoforms of CD44, such as CD44S, are found more expressed by both stem cells and mature cells, whereas the multiple CD44variant isoforms (CD44v) are found predominantly in stem cell subpopulations. In addition, there are 3 stages of BTSC subpopulations identified so far: stage 1 BTSCs express sodium iodide symporter (NIS), certain CD44v isoforms found also in stem cells, and CXCR4; they do not express LGR5 or EpCAM; stage 2 BTSCs express the particular isoforms of CD44variants found in stem cells, less of NIS but gain expression of LGR5 but not of EpCAM; stage 3 BTSCs (the only BTSCs found in the gallbladder and also found throughout the biliary tree) express LGR5 and EpCAM and a mix of CD44v and CD44s found in more mature cells. The stage 3 BTSCs are precursors to the hepatic stem cells progenitors and to the pancreatic stem cells.

The term “hepatic stem cells” (HpSCs) refers to stem cells found in the canals of Hering connecting the ends of the PBGs of the large intrahepatic bile ducts of the biliary tree to the plates of livercells. The HpSCs retain the ability to self-replicate and are multipotent. The biomarkers for these cells include epithelial cell adhesion molecule (EpCAM; found cytoplasmically and at the plasma membrane), neural cell adhesion molecule (NCAM), and very low levels (if any) of albumin, They express SOX9, SOX17, CD29 (ITBG1), HNF4-alpha, ONECUT2, low to moderate levels of one or more pluripotency genes (OCT4, SOX2, NANOG, KLF5, SALL4) and express cytokeratins 8, 18 and 19. They do not express PDX1 or alpha-fetoprotein (AFP) or P450-A7 or secretin receptor (SR).

The term “hepatoblasts” refers to bipotent hepatic stem cells that can give rise to hepatocytes and cholangiocytes. They have minimal ability to self-replicate under the conditions permissive for self-replication of the BTSCs and HpSCs. Still, they will extensively divide with treatment with additional cytokines and growth factors, but the divisions can include some degree of differentiation These cells are characterized by a biomarker profile that overlaps with but is distinct from HpSCs and distinct also from BTSCs. It includes expression of HNF4-alpha, CPS1, APOB, EpCAM (primarily at the plasma membrane), P450-A7, cytokeratin 7, 19, 8 and 18, secretin receptor, albumin, high levels of AFP, intercellular adhesion molecule (ICAM-1) but not NCAM, DLK1, and minimal (if any) pluripotency genes.

As used herein the term “committed progenitor” refers to a unipotent progenitor cell that gives rise to a single cell type, e.g. a committed hepatocytic progenitor cell (usually recognized by expression of albumin, AFP, glycogen, ICAM-1, various enzymes involved with glycogen synthesis) and gives rise to hepatocytes. The committed biliary (or cholangiocytic) progenitor (usually recognized by expression of EpCAM, cytokeratins 7 and 19, aquaporins, CFTR, membrane pumps associated with production of bile transport (bile salts are synthesized by hepatocytes) gives rise to cholangiocytes.

The descriptor “mature” when used to describe a cell refers to a differentiated cell. For example, “mature hepatocytes” refer to the dominant parenchymal cells in the liver that will be diploid in the periportal region, a mix of diploid and polyploid in the mid-acinar region, and mostly polyploid in the pericentral zone. The gene expression profile may be zonally lineage dependent and includes zone 1 genes (representative ones being transferrin mRNA (without an ability to undergo translation to a protein), connexin 28, and enzymes involved in glycogen synthesis), zone 2 genes (representative ones being tyrosine aminotransferase, transferrin mRNA that is able to undergo translation to a protein, and the highest level of expression of albumin), and zone 3 genes (representative ones being late P450s such as P450-3A4 and genes associated with apoptosis). See, e.g. Turner et al Human Hepatic Stem Cell and Liver Lineage Biology. Hepatology, 2011; 53: 1035-1045 (a more detailed listing of genes expressed in patterns associated with liver acinar zones), incorporated herein by reference. The final parenchymal cell layer in zone 3 consists of diploid, axin2+, unipotent hepatic progenitor cells that are connected to the endothelia of the central vein.

The term “angioblasts” is used to describe multipotent precursors giving rise to endothelia, stellate cells and to pericytes with associated mesenchymal stem cells. These cells may express one or more biomarkers such as CD117, VEGF-receptor, Van Willebrand factor, CD133. See. e.g., Geevarghese A. and Herman I., Transl Res. 2014; 163(4):296-306 (discussing overlap in biomarkers between mesenchymal lineages), incorporated herein by reference. The angioblasts may also give rise also to mesenchymal stem cells (MSCs) and thence to pericytes, forms of smooth muscle cells that are wrapped around the endothelia and in their contractility help to move blood from zone 1 through to zone 3 and then into the central vein. They produce numerous factors involved in vasculogenesis and that include hepatocyte growth factor (HGF), vascular endothelial growth factor (VEGF), endothelin, IGF II, epidermal growth factor (EGF), acidic fibroblast growth factor (a-FGF), and neurotrophins. See Geevarghese (2014), FIG. 13.

The term “stellate cell precursors” refers to unipotent precursors to stellate cells; one of the mesenchymal partners for hepatoblasts and the mesenchymal partner for committed cholangiocytic progenitors. Biomarkers for these cells include CD146 (also called Mel-CAM), alpha-smooth muscle actin and desmin. The stellate cell precursors are known to produce a wealth of paracrine signals needed for the hepatoblasts and for the committed progenitors and that include growth factors, such as hepatocyte growth factor (HGF) and stromal-derived growth factor (SDGF), and early lineage stage matrix components such as laminin and type IV collagen.

The term “endothelial cell precursors” refers to unipotent precursors to endothelia; the other mesenchymal partner for hepatoblasts and also the mesenchymal partner for committed hepatocytic progenitors. Biomarkers for these cells include VEGF-receptor, Van Willebrand factor, CD133, and CD31 (also called PECAM). These cells are known to produce paracrine signals that also include growth factors (e.g. VEGFs, angiopoietins) and matrix components (e.g. type IV collagen, laminin, and forms of heparan sulfate proteoglycans).

The term “mature stellate cells” is used to refer to the mesenchymal cell partners for cholangiocytes. The biomarkers for these cells include alpha smooth muscle actin and desmin, The mature stellate cells, but not the precursors, express significant levels of retinoids (vitamin A derivatives), glial fibrillary acidic protein (GFAP), type I and III collagen and other mature matrix components, and other markers of mature stellate cells as shown in the figure above.

The term “endothelial cells” is used to describe the mesenchymal cell partners for the hepatocytes. Their phenotypic traits transition from ones forming complete basement membranes with the hepatocytes near the portal triads to ones resulting in fenestrated (“windows”) endothelia with gaps between the cells and in the matrix with proximity to the central vein. The biomarkers include high levels of CD31 and the VEGF-receptor.

The term “hematopoietic cells” (this is the British term; the American term is hemopoietic) is a term of art that encompasses cells produced in the liver in fetal and perinatal stages and thereafter in the bone marrow, included but not limited to hemopoietic stem cells, lymphocytes, granulocytes, monocytes, macrophages, platelets, natural killer cells (called Pit cells in the liver), and erythrocytes.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

As used herein, the term “container” refers to an apparatus specifically configured to house cells and/or tissues. In some embodiments, such a container may be a bioreactor designed to accommodate a biomatrix scaffold. In further embodiments, the container may be configured for processing of decellularizing and/or recellularizing said scaffold.

The term “culture” or “cell culture” means the maintenance of cells in an artificial, in vitro environment, in some embodiments as adherent cells (e.g. monolayer cultures) or as floating aggregates cultures of spheroids or organoids. The term “spheroid” indicates a floating aggregate of cells all being the same cell type (e.g. an aggregate from a cell line); an “organoid” is a floating aggregate of cells comprised of multiple cell types. In some embodiments, this will be an epithelial cell and its mesenchymal partner cells, typically an endothelial cell and/or a stromal cell. The cells can be stem/progenitors of these categories of cells or can be mature cells. A “cell culture system” is used herein to refer to culture conditions in which a population of cells may be grown.

“Culture medium” is used herein to refer to a nutrient solution for the culturing, growth, or proliferation of cells. Culture medium may be characterized by functional properties such as, but not limited to, the ability to maintain cells in a particular state (e.g. a pluripotent state, a quiescent state, etc.), to mature cells—in some instances, specifically, to promote the differentiation of progenitor cells into cells of a particular lineage. Non-limiting examples of culture medium are Kubota's medium and Hormonally Defined Medium for Liver, which are further defined herein below. In some embodiments the medium may be a “seeding medium” used to present or introduce cells into a given environment. In other embodiments, the medium may be a “differentiation medium” used to facilitate the differentiation of cells. Such media may be comprised of a “basal medium” or a mixture of nutrients, minerals, amino acids, sugars and trace elements and may be used for maintenance of cells ex vivo.

More specifically, a “basal medium” is a buffer comprised of amino acids, sugars, lipids, vitamins, minerals, salts, and various nutrients in compositions that mimic the chemical constituents of interstitial fluid around cells. Such media may optionally be supplemented with serum to provide requisite signaling molecules (hormones, growth factors) needed to drive a biological process (e.g. proliferation, differentiation). Although the serum can be autologous to the cell types used in cultures, it is most commonly serum from animals routinely slaughtered for agricultural or food purposes such as serum from cows, sheep, goats, horses, etc. Media supplemented with serum may be optionally referred to as serum supplemented media (SSM).

Many of the commercially available forms of basal media are usable for epithelial stem/progenitor cells but must be modified to maintain stemness traits in the cells. Studies (Kubota et al, PNAS, 2000; 97(22): 12132-12137) have shown that to keep endodermal epithelial cells in an undifferentiated state, that is as stem cells, one may use a medium that is serum-free; with low oxygen levels (1-2%); devoid of copper; with an absence of cytokines and growth factors; with calcium levels below 0.5 mM; with supplements of insulin and transferrin/fe, with a mixture of purified free fatty acids that are complexed with a relevant carrier molecule such as albumin, and optimally (but not strictly required) a lipoprotein such as high density lipoprotein. Such an optimized medium for stem cells has been developed for endodermal stem cells, and is referred to as “Kubota's Medium,” defined hereinbelow. It enables the endodermal stem cells to expand in a self-replicative fashion for months. (Kubota and Reid PNAS 2000; 97(22): 12132-12137) The stability of the epithelial cells as stem cells may be optionally enhanced if the cells are cultured in Kubota's Medium and on substrata of hyaluronans or in hydrogels of hyaluronans or in the medium supplemented with hyaluronans. Y. Wang, H. L. Yao, C. B. Cui et al. Hepatology. 2010; 52(4):1443-54, U.S. Pat. No. 8,802,081 incorporated herein by reference.

The later maturational lineage stages of precursors, such as hepatoblasts and committed progenitors, have limited capacity to self-replicate but they have considerable ability to expand; the conditions for this expansion consists of supplementation of Kubota's Medium with various growth factors and cytokines such as HGF, EGF, forms of FGF, IL-6, IL-11 and others and use of matrix substrata that include type III and/or type IV collagen and laminin. (See, e.g., Kubota and Reid PNAS 2000; 97(22): 12132-12137; Turner et al; Journal of Biomedical Biomaterials. 2000; 82(1): pp. 156-168; Y. Wang, H. L. Yao, C. B. Cui et al. Hepatology. 2010 October 52(4):1443-54, incorporated by reference herein.)

As used herein, “differentiation” means that specific conditions cause cells to mature to adult cell types that produce adult specific gene products.

The terms “equivalent” or “biological equivalent” are used interchangeably when referring to a particular molecule, biological, or cellular material and intend those having minimal homology while still maintaining desired structure or functionality.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

The term “extracellular matrix,” or “ECM,” as used herein, refers to the complex scaffold comprised of various biologically active molecules secreted by cells, adjacent to one or more cell surfaces, and involved in the structural and/or functional support of cells and tissues or organs comprised thereof. Specific matrix components and concentrations thereof may be associated with specific tissue types, histological structures, organs, and other super-cellular structures. Components of the extracellular matrix relevant to the instant disclosure include, but are not limited to, collagens, collagen-associated matrix components, and growth factors.

Exemplary collagens include any and all types of collagen, such as but not limited to Type I through Type XXIX collagens. The biomatrix scaffold may comprise at least about 50%, 60%, 70%, 75%, 80%, 85%, 90%, 95%, 97%, 98%, 99%, 99.5% or more of one or more of the collagens found in the native biological tissue. In some embodiments the collagens are cross-linked and/or uncross-linked. The amount of collagen in the biomatrix scaffold can be determined by various methods known in the art and as described herein, such as but not limited to determining the hydroxyproline content. Exemplary methods of determining whether the cross-linked or uncross-linked character of a collagen also exist, such as those that rely on observing its dissolution properties. See e.g. D. R. Eyre,* M. Weis, and J. Wu. Advances in collagen cross-link analysis Methods, 2009; 45 (1): 65-74 (describing analysis of cross-linking by standard methods in the field of collagen chemistry). For example, a collagen may be determined to be cross-linked based on whether it dissolves in buffers at or below 1 M salt concentration.

Exemplary collagen-associated matrix components include, but are not limited to, adhesion molecules; adhesion proteins; L- and P-selectin; heparin-binding growth-associated molecule (HB-GAM); thrombospondin type I repeat (TSR); amyloid P (AP); laminins; nidogens/entactins; fibronectins; elastins; vimentins; proteoglycans (PGs); chondroitin sulfate PGs (CS-PGs); dermatan sulfate-PGs (DS-PGs); members of the small leucine-rich proteoglycans (SLRP) family such as biglycan and decorins; heparin-PGs (HP-PGs); heparan sulfate-PGs (HS-PGs) such as glypicans, syndecans, and perlecans; and glycosaminoglycans (GAGs) such as hyaluronans, heparan sulfates, chondroitin sulfates, keratin sulfates, and heparins.

In some embodiments the biomatrix scaffold comprises, consists of, or consists essentially of collagens, fibronectins, laminins, nidogens/entactins, elastins, proteoglycans, glycosaminoglycans (GAGs), growth factors, hormones, and cytokines (in any combination) bound to various matrix components. The biomatrix scaffold may comprise at least about 50%, 70%, 75%, 80%, 85%, 90%, 95%, 97%, 98%, 99%, 99.5% or more of one or more of the collagen-associated matrix components, hormones and/or cytokines found in the natural biological tissue and/or may have one or more of these components present at a concentration that is at least about 50%, 70%, 75%, 80%, 85%, 90%, 95%, 97%, 98%, 99%, 99.5% or more of that found in the natural biological tissue.

In some embodiments the biomatrix scaffold comprises all or most of the collagen-associated matrix components, hormones and/or cytokines known to be in the tissue. In other embodiments the biomatrix scaffold comprises, consists essentially of or consists of one or more of the collagen-associated matrix components, hormones and/or cytokines at concentrations that are close to those found in the natural biological tissue (e.g., about 50%, 60%, 70%, 75%, 80%, 85%, 90%, 95%, 98% or 100% of the concentration found in the natural tissue).

Exemplary matrix-bound signaling molecules include, but are not limited to, epidermal growth factors (EGFs), fibroblast growth factors (FGFs), hepatocyte growth factors (HGFs), insulin-like growth factors (IGFs), transforming growth factors (TGFs), nerve growth factors (NGFs), neurotrophic factors, interleukins, leukemia inhibitory factors (LIFs), vascular endothelial cell growth factors (VEGFs), platelet-derived growth factors (PDGFs), bone morphogenetic factors, stem cell factor (SCFs), colony stimulating factors (CSFs), GM-CSFs, erythropoietin, thrombopoietin, heparin binding growth factors, IGF binding proteins, placental growth factors, and Wnt signals.

Exemplary cytokines include, but are not limited to interleukins, lymphokines, monokines, colony stimulating factors, chemokines, interferons and tumor necrosis factor (TNF). The biomatrix scaffold may comprise at least about 20%, 30%, 40%, 50%, 60%, 70%, 75%, 80%, 85%, 90%, 95%, 97%, 98%, 99%, 99.5%, 100% or more (in any combination) of one or more of the matrix bound growth factors and/or cytokines found in the natural biological tissue and/or may have one or more of these growth factors and/or cytokines (in any combination) present at a concentration that is at least about 20%, 30%, 40%, 50%, 60%, 70%, 75%, 80%, 85%, 90%, 95%, 97%, 98%, 99%, 99.5%, 100% or more of that found in the natural biological tissue.

In some embodiments the biomatrix scaffold comprises physiological levels or near-physiological levels of many or most of the matrix bound growth factors, hormones and/or cytokines known to be in the natural tissue and/or detected in the tissue and in other embodiments the biomatrix scaffold comprises one or more of the matrix bound growth factors, hormones and/or cytokines at concentrations that are similar to or close to those physiological concentrations found in the natural biological tissue (e.g., differing by no more than about 50%, 40%, 30%, 25%, 20%, 25%, 20%, 15%, 10%, 5%, 4%, 3%, 2%, 1%, 0.5% in comparison). The amount or concentration of growth factors or cytokines present in the biomatrix scaffold can be determined by various methods known in the art and as described herein, such as but not limited to various antibody assays and growth factor assays.

As used herein, the term “functional” may be used to modify any molecule, biological, or cellular material to intend that it accomplishes a particular, specified effect.

The term “gene” as used herein is meant to broadly include any nucleic acid sequence transcribed into an RNA molecule, whether the RNA is coding (e.g., mRNA) or non-coding (e.g., ncRNA).

As used herein, the term “generate” and its equivalents (e.g. generating, generated, etc.) are used interchangeable with “produce” and its equivalents when referring to the method steps that bring the micro-organ or engineered tissue of the instant disclosure into existence.

“Hormonally Defined Medium for Liver” or “HDM-L” as used herein comprises classic factors for differentiation of the stem cells to mature cells; such media are generally comprised of basal media supplemented with a mixture of hormones, growth factors, and various nutrients and utilized serum-free for expansion or differentiation of specific cell types—e.g. parenchymal cells. In some embodiments, it may be prepared by supplementing Kubota's medium, which is defined for stem cells, with additional hormones and factors needed for differentiation of the cells. Exemplary growth factors for use in such a differentiation medium are disclosed in Y. Wang, H. L. Yao, C. B. Cui et al. Hepatology. 2010 October 52(4):1443-54 and U.S. Pat. No. 8,404,483 incorporated herein by reference in its entirety. Aspects of this disclosure relate to a specific HDM-L designated “BIO-LIV-HDM” throughout the experiments designed to differentiate stem cells and progenitors of both parenchymal and non-parenchymal lineages and/or epithelial and mesenchymal lineages to yield mature liver tissue. In addition, the BIO-LIV-HDM-L was supplemented further with growth factors and hormones required for the various non-parenchymal cell types including the mesenchymal cell (stellate cells, pericytes, endothelial), both precursor and mature forms, the neuronal cells, both precursors and mature forms, and the hematopoietic cells, both precursors and mature forms.

As used herein, the term “hyaluronan,” or “hyaluronic acid,” refers to a polymer of a uronic acid and an aminosugar [1-3] composed of a disaccharide unit of glucosamine and gluronic acid linked by β1-4, β1-3 bonds and salts thereof. Thus, the term hyaluronan refers to both natural and synthetic hyaluronans.

“Hydrogel” used herein is intended to mean a three dimensional network formed by polymer chains retaining a significant fraction of an aqueous medium within said three dimensional network without dissolving in said aqueous medium.

The term “isolated” as used herein refers to molecules or biologicals or cellular materials being substantially free from other materials.

“Kubota's medium” as used herein refers to a serum-free, hormonally defined medium designed for endodermal stem cells and enabling them to expand clonogenically in a self-replicative mode of division (for example, on hyaluronan substrata or in buffers containing hyaluronans). Kubota's may refer to any basal medium containing no copper, low calcium (<0.5 mM), insulin, transferrin/Fe, a mix of purified free fatty acids bound to purified albumin and, optionally, also high density lipoprotein. Kubota's Medium or its equivalent is serum-free and contains only a purified and defined mix of hormones, growth factors, and nutrients. In certain embodiments, the medium is comprised of a serum-free basal medium (e.g., RPMI 1640 or DME/F12) containing no copper, low calcium (<0.5 mM) and supplemented with insulin (5 μg/mL), transferrin/Fe (5 μg/mL), high density lipoprotein (10 μg/mL), selenium (10⁻¹⁰ M), zinc (10¹² M), nicotinamide (5 μg/mL), and a mixture of purified free fatty acids bound to a form of purified albumin. Non-limiting, exemplary methods for the preparation of this media have been published elsewhere, e.g., Kubota H, Reid L M, Proceedings of the National Academy of Sciences (USA) 2000; 97:12132-12137, Y. Wang, H. L. Yao, C. B. Cui et al. Hepatology. 2010; 52(4):1443-54, Turner et al; Journal of Biomedical Biomaterials. 2000; 82(1): pp. 156-168; Y. Wang, H. L. Yao, C. B. Cui et al. Hepatology. 2010 October 52(4):1443-54, the disclosures of which is incorporated herein by reference. Kubota's Medium may be designed for specific cell types by providing specific factors and supplements to allow for specific expansion under serum free conditions. For example, Kubota's Medium modified for use with hepatoblasts is designed for hepatoblasts and their descendants, committed progenitors, and promotes their expansion under serum-free conditions. The expansion might occur with self-replication but usually occurs with minimal (if any) self-replication. The medium is especially effective if the cells are on substrata of type IV collagen and laminin.

The terms “nucleic acid,” “polynucleotide,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers.

A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “organ” a structure which is a specific portion of an individual organism, where a certain function or functions of the individual organism is locally performed and which is morphologically separate. Non-limiting examples of organs include the skin, blood vessels, cornea, thymus, kidney, heart, liver, umbilical cord, intestine, nerve, lung, placenta, pancreas, thyroid and brain. Organs may be used as a tissue source, for example, fetal, neonatal, pediatric, child, or adult organs may be used to derive cell populations of interest for uses disclosed herein.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “subject” is intended to mean any animal. In some embodiments, the subject may be a mammal; in further embodiments, the subject may be a human, mouse, or rat.

The term “tissue” is used herein to refer to tissue of a living or deceased organism or any tissue derived from or designed to mimic a living or deceased organism. The tissue may be healthy, diseased, and/or have genetic mutations. The term “natural tissue” or “biological tissue” and variations thereof as used herein refer to the biological tissue as it exists in its natural or in a state unmodified from when it was derived from an organism. A “micro-organ” refers to a segment of “bioengineered tissue” that mimics “natural tissue.”

The biological tissue may include any single tissue (e.g., a collection of cells that may be interconnected) or a group of tissues making up an organ or part or region of the body of an organism. The tissue may comprise a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

### II. ABBREVIATIONS

Portions of this disclosure utilize acronyms to refer to certain terms. Acronyms for cell populations may be referred to herein by a small letter to indicate the species: r=rat; m=murine; h=human. If an acronym for a molecule is printed in Italics, it refers to the gene; if in regular font, then it refers to the protein encoded by the gene

The following is a non-limiting list of abbreviations used herein: ACOX, acyl-coenzyme A oxidase; APOL6, Apolipoprotein L6; AFP, α-fetoprotein, a signature gene expressed by hepatoblasts; ASMA, α-smooth muscle actin; ALB, albumin; ALT, alanine aminotransferase; AST, aspartate aminotransferase; ccK18, cleaved caspase K18, when secreted, an indicator of cell necrosis; C/EBP, CCAAT/enhancer-binding protein alpha; CD, common determinant; CD31, platelet endothelial cell antigen (or PECAM), a surface marker of endothelial cells; CD34, hemopoietic stem/progenitor cell antigen; CD45, common leucocyte antigen found on most hemopoietic cell subpopulations; CD133, prominin, a surface marker found on endothelial and parenchymal cell precursors; CSF, Colony stimulating factor; CYP, cytochrome P450 mono-oxygenases that catalyze many reactions associated with drug metabolism and/or synthesis of cholesterol, steroids and lipids; There are forms expressed in early lineage stages (CYP3A7 and possibly CYP1B1) and others late lineage stages of parenchymal cells (e.g. CYP 1A1, CYP2C8, CYP3A4); CK, cytokeratin; CK7, cytokeratin associated with biliary cells; CK8 and 18, cytokeratins associated with all epithelia; EGF, epidermal growth factor; EpCAM, epithelial cell adhesion molecule; FBS, fetal bovine serum; FGF, fibroblast growth factor; bFGF, basic fibroblast growth factor; GAGs, glycosaminoglycans, carbohydrate chains that are polymers of a dimer (uronic acid and an amino sugar), most of them with specific sulfation patterns, and that play diverse roles cooperatively with proteins in signal transduction processes; GATA, transcription factors with a zinc binding DNA binding domain to the DNA sequence, GATA; GATA-2, GATA binding protein 2, a regulator of hematopoietic gene expression; HBs, hepatoblasts; HDL, high-density lipoprotein; hGH, human growth hormone; HGF, hepatocyte growth factor; HpSCs, hepatic stem cells; HDM, hormonally defined medium; H&E, hematoxylin and eosin; HNF, hepatocyte nuclear factor; HNF1a, hepatocyte nuclear factor homeobox A expressed in all hepatic parenchymal precursors; HNF1b, hepatocyte nuclear factor homeobox B, found involved developmentally in hepato-pancreatic specification; HPLC, High-performance liquid chromatography; IGF, insulin-like growth factors that share homologies with insulin and that act either as mitogens or differentiation signals depending on the specific GAGs with which they are associated; IGF-I, insulin-like growth factor I, well known as a key regulator in adult liver cells; IGF-II, insulin-like growth factor II, a major regulator in fetal liver cells; IL, interleukin; IL7-R, receptor for interleukin 7, critical in the development of lymphocytes; JAG1, Jagged1, also called CD339, a key gene in the notch signaling pathway involved in fate determination; K18, total cytokeratin 18, if released by cells indicates cell death or necrosis; KM, Kubota's Medium; LGR5, Leucine-rich repeat-containing G-protein coupled receptor 5, an important stem cell marker in intestine, liver and pancreas; LDH, lactate dehydrogenase; LDLR, Low-Density Lipoprotein (LDL) Receptor; LYVE-1, lymphatic endothelial hyaluronan cell receptor; MST1, macrophage stimulating 1; MMP, matrix metalloproteinase (or peptidase); MMP2, matrix metalloproteinase-2, the 72 kDa type IV collagenase or gelatinase A (GELA); MMP9, matrix metallopeptidase 9, also known as 92 kDa type IV collagenase or gelatinase B (GELB), is a matrixin, a class of enzymes of the zinc-metalloproteinases family involved in degradation of the extracellular matrix; MRP2, Multidrug resistance-associated protein 2; NMR, Nuclear Magnetic Resonance; PAR, protease activated receptor, PAS, Periodic acid-Schiff; PDGF, platelet-derived growth factor; RAG1, Recombination activating gene 1; SEM, scanning electron microscopy; SCF, stem cell factor; SCTR, secretin receptor; SLC4A2, Solute carrier family 4 (anion exchanger), member 2; TGF, transforming growth factor; TEM, transmission electron microscopy; VEGF, vascular endothelial cell growth factor.

### III. MODES FOR PRACTICING THE PRESENT DISCLOSURE

Aspects of the disclosure relate to compositions and methods for producing a bioengineered tissue and a container configured for the generation thereof.

Specific embodiments relate to a method for the generation of bioengineered tissue comprising (a) introducing a suspension of cells in a seeding medium into or onto a biomatrix scaffold and (b) replacing the seeding medium with a differentiation medium after an initial incubation period. In some embodiments, this method is carried out in a container specifically designed for execution of such a process. Aspects of the disclosure relate to the container. In some embodiments, this container is configured with a flow path specifically designed to mimic vascular support of cells. In further embodiments, this may be achieved through the use of a three-dimensional biomatrix scaffold comprising a matrix remnant of the vascular tree.

In some embodiments, the seeding occurs in multiple intervals followed by a period of rest; these intervals and rest periods may vary in duration from about 1 to about 15 minutes, e.g. about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, and/or 15 minutes. The number of cells introduced and the concentration thereof may likewise be varied. For example, in some embodiments, about 10 to 12 million cells per gram wet weight scaffold may be introduced over a given interval. In some embodiments, the rate of introduction may be at 15 mL/minute for a given number of intervals—one, two, three, four, or more intervals—and then reduced to a rate of, for example 1.3 mL/min after the given number of intervals.

In some embodiments, the cells and seeding medium may be pre-incubated before introduction, e.g. at 4° C. for 4 to 6 hours.

In some embodiments, the biomatrix scaffold may be derived from a specific organism, which may be the same or different from the organism from which the progenitor cells are derived.

In some embodiments, a biomatrix scaffold may be prepared from a biological tissue by perfusing a biological tissue sample with multiple buffers and rinse media to decellularize the tissue to retain only or primarily the extracellular matrix components yielding a scaffold of the matrix from the tissue and that maintains the intrastructure of the tissue's histology. In alternate embodiments, an intact biomatrix scaffold may be obtained from a commercially available source.

A culture medium acceptable for the generation of the bioengineered tissue may be selected based on the desired characteristics of the tissue, e.g. cultures may be selected on the presence of certain factors that stimulate the differentiation and/or growth of the population of progenitor cells into cells of a particular organ or tissue type, such as those described in Y. Wang, H. L. Yao, C. B. Cui et al. Hepatology. 2010 October 52(4):1443-54, incorporated herein by reference in its entirety. Further, at different stages in the generation of the process of generating the bioengineered tissue, different media may be relevant—e.g. a seeding medium or a differentiation medium.

Further disclosures regarding the use of factors and other media components to achieve a specific outcome are disclosed in U.S. application Ser. No. 12/213,100 and U.S. Pat. No. 8,404,483, which are incorporated herein by reference in their entirety. In certain embodiments, the culture medium is a medium that promotes cell differentiation.

In some embodiments, the medium further comprises one or more cell growth or differentiation factors, such as those described herein above.

In some embodiments, the seeding medium comprises one or more of: calcium at a concentration between about 0.3 mM to 0.5 mM, trace elements (such as selenium and zinc but not copper, a mixture of purified free fatty acids (such as palmitic acid, palmitoleic acid, stearic acid, oleic acid, linoleic acid, linolenic acid), one or more lipid binding proteins (such as HDL), insulin, and transferrin/fe. In some embodiments, the seeding medium comprises serum, optionally used to inactivate enzymes used in preparing cell suspensions. A non-limiting example of such serum is fetal bovine serum (FBS). In some embodiments where serum is added, it is replaced with a serum free medium (e.g. serum free HDM), typically within about 6 hours to 24 hours and/or as soon as possible.

In some embodiments, the differentiation medium comprises one or more of: calcium at a concentration of at least about 0.5 mM, trace elements, ethanolamine, glutathione, ascorbic acid, minerals, amino acids, and sodium pyruvate, a mixture of purified free fatty acids, one or more lipid binding proteins (such as HDL), one or more sugars, one or more glucocorticoids, insulin, transferrin f/e, one or more hormones and/or growth factors—such as, but not limited to, those for the propagation and/or maintenance of parenchymal cells (prolactin, growth hormone, glucagon, and thyroid hormones (e.g. tri-iodothryronine or T3), epidermal growth factors (EGFs), hepatocyte growth factors (HGFs), fibroblast growth factors (FGFs), insulin like growth factors (IGFs), bone morphogenetic proteins, Wnt ligands, and cyclic adenosine monophosphate) and/or those for the propagation and/or maintenance of non-parenchymal cells (angiopoietin, vascular endothelial cell growth factors (VEGFs), nerve growth factor, stem cell factor, leukemia inhibitory factor (LIF), colony stimulating factors (CSFs), thrombopoietin, platelet derived growth factors (PDGFs), erythropoietin, insulin-like growth factors (IGFs) fibroblast growth factors (FGFs), and epidermal growth factors (EGFs)).

In some embodiments, the suspension of cells may be derived from a specific organism, which may be the same or different from the organism from which the biomatrix scaffold is derived. Stem or progenitor cells may be obtained from commercially available sources including but not limited to direct commercial retailers or repositories such as the America Type Culture Collection (ATCC, http://www.atcc.org/). Alternatively, methods of generating and/or isolating stem or progenitor cells from samples are disclosed in the art. Exemplary methods include those disclosed in U.S. application Ser. No. 12/926,161 incorporated herein by reference in its entirety. Non-limiting exemplary sources of cells include the liver, biliary tree, gallbladder, hepato-pancreatic common duct, pancreas, duodenum, bone marrow, and endothelia (e.g. hepatic or biliary tree stem cells from the biliary tree or gallbladder, bone marrow stem cells, and endothelial stem cells). Further examples include embryonic stem (ES) cells or induced pluripotent stem (iPS) cells from any source.

In some embodiments, the population of suspension cells may be a homogenous population of cells—comprising only cells of the same type—or a heterogeneous population of cells—comprising cells of different types. The number and concentration of cells in the population of suspension of cells cultured may be determined based on the suspension cells, the culture medium, the culture size, the desired organ/tissue characteristics, and other factors of relevance. In some embodiments, the number of cells in the population of progenitor cells is determined by the growth rate and differentiation conditions of the stem/progenitor cells. In some embodiments, the number of cells in the population of stem/progenitor cells is determined by the growth factors and other components present in the culture medium.

In some embodiments, the suspension of cells comprises parenchymal cells (e.g. BTSCs, HpSCs, hepatoblasts, pancreatic stem cells, hepatic or pancreatic committed progenitors, hepatocytes, cholangiocytes, islets, acinar cells) and non-parenchymal cells, wherein the non-parenchymal cells include subpopulations of mesenchymal cells (e.g. angioblasts or precursors of stellate cells or of endothelia, mature stellate or mature endothelial cells), neuronal precursors and mature neuronal cells, and hematopoietic precursors and mature hematopoietic cells (e.g. precursors of lymphocytes, myeloid cells, natural killer cells, platelets, erythrocytes or their mature counterparts). In some embodiments, these cells are in a ratio of about 10%/90%, 20%/80%, 30%/70%, 40%/60%, 50%/50%, 60%/40%, 70%/30%, 80%/20%, and 10%/90%. In some embodiments, the suspension of cells may comprise at least about 50% precursor and/or stem cells. In some embodiments, the cell suspension comprises no terminally differentiated hepatocytes.

In some embodiments, the gene or protein expression of the culture may be monitored over the time sufficient to generate the bioengineered tissue. In certain embodiments, the gene or protein expression profile of the cultured population of progenitor cells at a specific time point may be compared to the gene or protein expression profile of a population of cells selected from (i) the cultured population of progenitor cells at an earlier or later time point, (ii) a control sample population of progenitor cells, (iii) a population of differentiated cells derived from an organ or tissue. Similarly, histology of the tissue may be compared to earlier or later stages of development of the desired target tissue.

Not to be bound by theory, it is envisioned that over the time sufficient to generate the bioengineered tissue, the gene or protein expression profiles and/or histology of the cultured cells will shift to resemble that of a population of differentiated cells derived from an organ or tissue or less differentiated precursors thereof.

Aspects of the disclosure relate to the three-dimensional biomatrix scaffold comprising a matrix remnant of the vascular tree of the organ from which the scaffold is derived. In some embodiments, the scaffold also comprises native collagens found in the organ from which the scaffold is derived.

A further aspect of the disclosure relates to a bioengineered tissue and/or micro-organ produced using the compositions and methods disclosed herein. In some embodiments, the resulting tissue demonstrates the maturationally lineage-dependent or zonation dependent phenotypic characteristics of native liver, such as, but not limited to, (a) periportal region, (b) a region having sinusoidal plates of parenchymal cells and mesenchymal cells. The phenotypic traits may further include periportal traits associated with diploid cells, traits of mature parenchymal and mesenchymal cells found in the mid-acinar region of native liver, traits of parenchymal and mesenchymal cells of the pericentral zone. The bioengineered tissue and/or micro-organ may further comprise (i) polyploid hepatocytes associated with fenestrated endothelial cells and/or (ii) diploid hepatocytes connected to endothelia of a central vein and/or cholangiocytes associated with stellate cells. If the bioengineered tissue and/or micro-organ is designed for pancreas, then it may further comprise acinar and islet cells.

In some embodiments, the periportal region of the bioengineered tissue and/or micro-organ is enriched in traits of the stem/progenitor cell niches that comprise hepatic stem cells, hepatoblasts and committed progenitors. In some embodiments, the parenchymal cells of the bioengineered tissue and/or micro-organ further comprise young (diploid) hepatocytes and cholangiocytes. In some embodiments, the mesenchymal cells of the bioengineered tissue and/or micro-organ of the periportal zone further comprise precursors of stellate cells, pericytes, smooth muscle cells and endothelia. In some embodiments, the mid-acinar region of the bioengineered tissue and/or micro-organ is enriched in traits of the mature parenchymal cells that comprise mature hepatocytes and cholangiocytes. In some embodiments, the parenchymal cells of the bioengineered tissue and/or micro-organ further comprise hepatocytes and cholangiocytes. In some embodiments, the mesenchymal cells of the bioengineered tissue and/or micro-organ of the periportal zone further comprise stellate cells, pericytes, smooth muscle cells, neuronal cells, and endothelia. In some embodiments, the pericentral region of the bioengineered tissue and/or micro-organ is enriched in traits of the mature parenchymal cells, hepatocytes, expressing late genes such as late P450s (e.g. P450-3A), some of which are polyploid and some are undergoing apoptosis. In some embodiments, the mesenchymal cells of the pericentral zone of bioengineered tissue and/or micro-organ further comprises fenestrated endothelia.

In some embodiments, the bioengineered tissue and/or three-dimensional micro-organ disclosed herein may be useful for use in vivo or ex vivo. Non-limiting examples of potential uses include research uses for studying tissue morphogenesis, cell migration, clonal lineages, cell fate potential, cross species developmental timing, and cell-type specific genome expression; use of organoids as a model for high-throughput drug screening for a specific organ, cell replacement therapy, or other types of organ specific treatment; and transplantation.

Aspects of the disclosure also provide for kits comprising the appropriate container and/or media for the production of the bioengineered tissue or micro-organ. In further embodiments, the kit may further comprise instructions as to how to generate a bioengineered tissue or micro-organ.

### IV. EXAMPLES

The following examples are non-limiting and illustrative of procedures which can be used in various instances in carrying the disclosure into effect. Additionally, all reference disclosed herein below are incorporated by reference in their entirety.

Reagents and supplies for the investigations disclosed herein below were obtained from the following companies: Abcam, Cambridge, Mass.; ACD Labs, Toronto, CA; Acris Antibodies, Inc., San Diego, Calif.; Advanced Bioscience Resources Inc. (ABR), Rockville, Md.; Agilent Technologies, Santa Clara, Calif.; Alpco Diagnostics, Salem, N.H.; BD Pharmingen, San Jose, Calif.; Becton Dickenson, Franklin Lakes, N.J.; Bethyl Laboratories, Montgomery, Tex.; BioAssay Systems, Hayward, Calif.; Cambridge; Isotope Laboratories, Tewksbury, Mass.; Carl Zeiss Microscopy, Thornwood, N.Y.; Carolina Liquid Chemistries, Corp., Winston-Salem, N.C.; Charles River Laboratories International, Inc., Wilmington, Mass.; Chenomx, Alberta, Canada; Cole-Parmer, Court; Vernon Hills, Ill.; DiaPharma, West Chester Township, Ohio; Fisher Scientific, Pittsburgh, Pa.; Gatan, Inc., Pleasanton, Calif.; Illumina, San Diego, Calif.; Ingenuity, Redwood City, Calif.; Life Technologies Corp., Grand Island, N.Y.; LifeSpan Biosciences, Inc., Seattle, Wash.; Molecular Devices, Sunnyvale, Calif.; Olympus Scientific Solutions Americas Corp., Waltham, Mass.; Polysciences, Inc., Warrington, Pa.; Research Triangle Labs (TRL), Research Triangle Park, N.C.; R&D Systems, Minneapolis, Minn.; RayBiotech, Norcross, Ga.; Santa Cruz Biotechnology, Inc., Dallas, Tex.; Sigma Aldrich, St. Louis, Mo.; Tousimis Research Corp., Rockville, Md.; Qiagen, Germantown, Md.; Umetrics, Umea, Sweden

### Example 1—Human Liver Cell Sourcing and Processing

Human fetal livers were obtained by elective terminations of pregnancy and provided by an accredited agency, ABR. Tissues used in the experiments were from fetuses between 17-19 weeks. The research protocol was reviewed and approved by the Institutional Review Board (IRB) for Human Research Studies at the University of North Carolina at Chapel Hill. The method of preparation of human fetal liver cell suspensions was described in prior publications. Briefly, livers were first mechanically homogenized and then enzymatically dispersed into a cell suspension of RPMI-1640 supplemented with 0.1% bovine serum albumin (BSA), 1 nM selenium, 300 U/ml type IV collagenase, 0.3 mg/ml deoxyribonuclease and antibiotics. Digestion was done at 32° C. with frequent agitation for 30-60 minutes. Most tissues require two rounds of digestions followed by centrifugation at 1100 rpm at 4° C. Cell pellets were combined and resuspended in cell wash (RPMI-1640 with 0.1% BSA, 1 nM selenium and antibiotics). The cell suspension is centrifuged at 300 rpm for 5 minutes at 4° C. to remove red blood cells. The cell pellets were again resuspended in cell wash and filtered through a 70 m nylon cell strainer (Becton Dickenson). Aliquots of 1×106 cells were isolated and processed for RNA and used as a control for assays using qRT-PCR (t=0).

Adult human tissue (n=3) was obtained from Triangle Research Laboratories (TRL) either as flash frozen tissue, and were used as controls for mRNA expression via RNA-sequencing. Cells were processed for RNA using Qiagen RNeasy Mini Kit (Qiagen) per the manufacturer's instructions. Results from 3 donors were averaged for comparisons between fetal liver stem/progenitor cells (t=0) and bioreactors (t=14 days). Freshly isolated suspensions of adult human hepatocytes were obtained from TRL for the purpose of comparing traditionally used hepatocyte culture medium to hormonally defined, serum-free medium (HDM) designed for hepatic differentiation in the bioreactor experiments (BIO-LIV-HDM). Three plates of 6-well sandwich cultures, 1 plate per human donor, were cultured for 7 days under two different medium conditions. Triplicates of the cultures in each condition were prepared from each donor.

### Example 2—Preparation and Analysis of Biomatrix Scaffolds

Decellularization of rat livers. Wistar rats (weights 250-300 g) were obtained from Charles River Laboratories and housed in animal facilities handled by the UNC Division of Laboratory Animal Management. They were fed ad libitum until used for experiments. All experimental work was approved by and performed in accordance with the UNC Institutional Animal Use and Care Committee guidelines.

The protocol for decellularizing livers to produce biomatrix scaffolds has been described previously. Wang Y., et al. (2011) Hepatology 53:293-305; Gessner, R. C. et al. (2013) Biomaterials 34:9341-9351. Male rats were anesthetized with Ketamine-Xylazine, and their abdominal cavity opened. The portal vein was cannulated with a 20-gauge catheter to provide a perfusion inlet to the vasculature of the liver, and the vena cava and hepatic artery were transected to provide an outlet for perfusion. The liver was removed from the abdominal cavity and placed in a perfusion bioreactor. The blood was removed by flushing the liver with 300 ml of serum-free DMEM/F12 (Gibco). This was followed by perfusion for 90 minutes with a high salt buffer (NaCl); solubility constants for known collagen types in liver are such that 3.4 M NaCl is adequate to keep them all in an insoluble state, along with any matrix components and cytokine/growth factors bound to the collagens or the collagen-bound matrix components. The liver was rinsed for 15 minutes with serum-free DMEM/F12 to eliminate the delipidation buffer and then followed by perfusion with 100 mls of DNase (1 mg per 100 mL; Fisher) and RNase (5 mgs per 100 mL; Sigma) to remove any residual nucleic acid contaminants. The final step was to rinse the scaffolds with serum-free DMEM/F12 for 1 hour to eliminate any residual salt or nucleases. Images are provided in FIG. 11. The biomatrix scaffolds were perfused at 1.3 ml/min via a Masterflex peristaltic pump (Cole-Parmer) for 2 hours with Kubota's medium supplemented with 10% fetal bovine serum (FBS) to prime the scaffold for cell seeding. Fetal liver cells were immediately seeded following priming. This step of using a SSM for priming the scaffolds can be eliminated if the cell suspension has been adequately treated to eliminate enzymes used in preparation of the cell suspension.

Collagen Analysis.

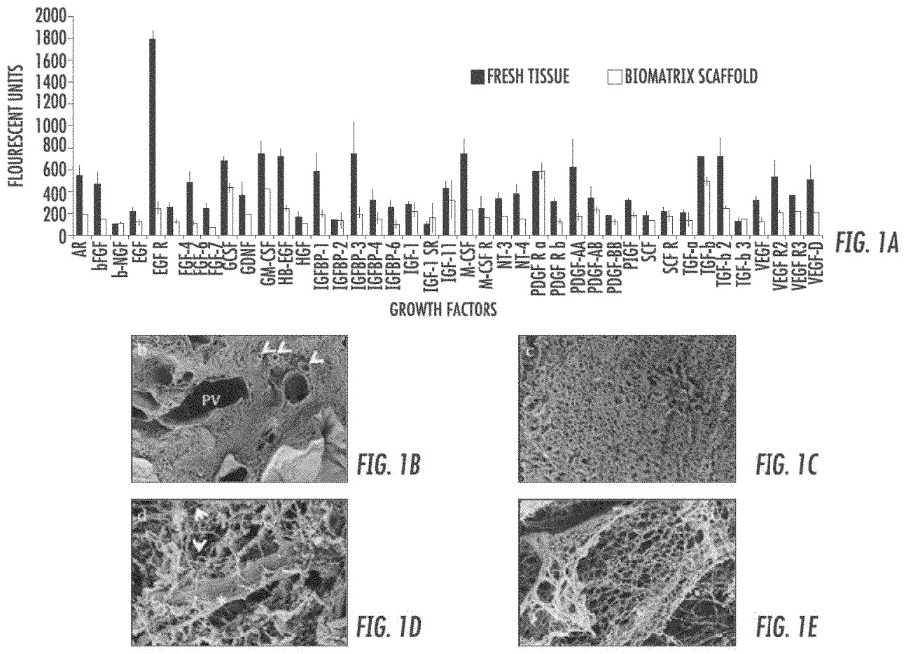

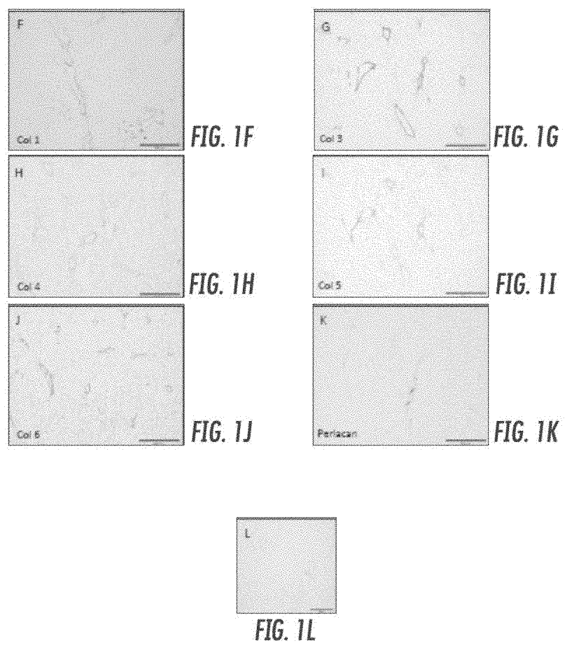

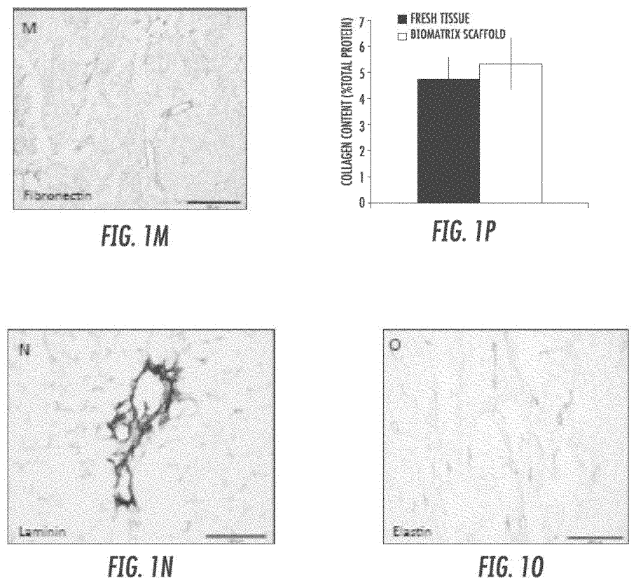

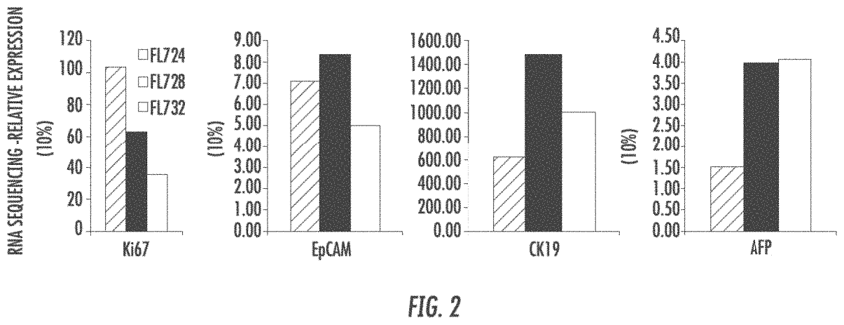

The amount of collagen in the biomatrix scaffolds was evaluated based on the hydroxyproline (hyp) content. Samples of fresh livers (n=5) and of biomatrix scaffolds (n=6) were flash frozen and pulverized into a powder. High-performance liquid chromatography (HPLC) was used to quantify the collagen content per total protein, and total collagen was estimated based on the hydroxyproline value of 300 residues/collagen. Assays were measured individually with a Cytofluor Spectramax 250 multi-well plate reader (Molecular Devices). Hydroxy-proline content was used to evaluate the extent of collagen retention following decellularization. These analyses were performed using HPLC to compare the amount of collagen from fresh tissue versus from biomatrix scaffolds (decellularized tissue). Results are presented as mass of hydroxyl-proline (an amino acid specific to collagen proteins). It was determined that ˜99% of all collagens were present following the decellularization of the rat liver (FIG. 1p).

Immunohistochemistry of Biomatrix.

Biomatrix scaffolds were embedded in OCT and flash frozen for frozen sectioning. Frozen sections were thawed for 1 hour at room temperature and then fixed in 10% buffered formaldehyde. After fixation, sections were washed 3 times in 1× phosphate buffered saline (PBS), followed by blocking of endogenous peroxidase with 3% H₂O₂ for 15 minutes at room temperature. After washing with 1×PBS, sections were again blocked with 2.5% horse serum in PBS for 1 hour at room temperature. Primary antibodies diluted in 2.5% horse serum in PBS were added and incubated overnight at 4° C. The next morning, sections were rinsed 3 times with PBS and incubated with secondary antibodies for 30 minutes at room temperature. The Nova Red substrate (Vector) was used as the developer, prepared according to manufacturer instructions. Images were taken using an Olympus IX70 microscope (Olympus). Hematoxylin and Eosin staining of the biomatrix scaffold revealed no remaining cells following decellularization (data not shown). Further analysis of the DNA/RNA content of the biomatrix scaffolds following decellularization was perform, and it was determined that the DNA/RNA levels were negligible.

Histology indicated the presence of collagens I, III, IV, V and VI to be present and in their traditional locations across the acinus (FIG. 1f-j). The high osmolarity maintained during the decellularization process keeps the collagens insoluble, and they are, therefore, present in the biomatrix scaffolds. Alcian blue staining also indicated qualitatively that proteoglycans, major components of the extracellular matrix, were also present (FIG. 1k,l); they are known as chemical scaffolds for growth factors and cytokines and influence the availability and activity of these factors. Basement membrane cell adhesion molecules (elastin, fibronectins and laminins) were identified in the appropriate zonal positions following decellularization (FIG. 1m-o). Both elastin and laminins were found in the periportal region where the hHpSCs and other hepatic precursors reside. Fibronectin was identified throughout the matrix, across all zones.

Growth Factors.

Samples of rat livers (fresh tissue) and rat liver biomatrix scaffolds (decellularized tissue) were analyzed for the presence and the concentration of matrix-bound growth factors and cytokines. The samples were flash-frozen in liquid nitrogen, pulverized at liquid nitrogen temperature into a powder and sent for analysis to RayBiotech. Semi-quantitative growth factor assays were done using the RayBiotech® Human Growth Factor Arrays G1 Series (Raybiotech) and results were reported in fluorescent intensity units (FIUs). The FIUs levels were reduced by the findings from negative controls for non-specific binding and normalized to protein concentration. Forty growth factors were assayed in fresh, non-decellularized rat liver tissue (n=3) and compared to those in biomatrix liver scaffolds (n=3). The data from the replicates were averaged. Although the assay was developed for human growth factors, there is sufficient overlap in cross-reactions to rat growth factors to permit use for rat tissue. Three samples of both fresh tissue and biomatrix scaffolds were analyzed for 40 growth factors (FIG. 1a). Analyses revealed that all of the growth factors found in the tissue in vivo remained with the biomatrix scaffold extracts; although most of them were at levels lower than in vivo, they were still at levels sufficient to be physiologically relevant. Of particular importance was the presence of growth factors associated with angiogenesis, such as multiple forms of FGF, PDGF and VEGF; and those important for cell proliferation and differentiation such as EGFs, heparin binding EGF, HGF, IGF I and II and their binding proteins, and TGF. The availability of these growth factors is important for many different biological functions (mitosis as well as tissue-specific gene expression).

Scanning Electron Microscopy (SEM).

Imaging of decellularized liver biomatrix revealed that there was retention of vasculature structures of native liver, including intact portal triads (FIG. 1b). Shown in FIG. 1b, bile ducts, the hepatic artery and portal vein are all evident. In addition, the honeycomb structures that would normally accommodate hepatic parenchyma were left intact but void of cells (FIG. 1c). Matrix molecules such as elastin, collagen I and III were also identifiable by SEM (FIG. 1d, e).

### Example 3—Media

All media were sterile-filtered (0.22 m filter) and kept in the dark at 4° C. before use. Basal medium and fetal bovine serum (FBS) were purchased from GIBCO/Invitrogen. All growth factors were purchased from R&D Systems. All other reagents, except those noted, were obtained from Sigma. Traditional hepatocyte maintenance medium (HMM), used in medium comparison studies, was purchased from Triangle Research Laboratories (TRL) and contained William's E medium supplemented with HEPES, GlutaMax, ITS+ (insulin, transferrin and selenium), dexamethasone, and penicillin-streptomycin.

Seeding Medium.

Kubota's medium, is a wholly defined, serum-free medium designed for clonogenic, self-replicative expansion of endodermal stem/progenitors. It was used serum-free for monolayer cultures or organoid cultures of fetal liver cells. Kubota's medium has been shown effective in culture selection of murine, rodent and human hepatic stem/progenitors. This medium consists of RPMI-1640 with no copper, low calcium (0.3 mM), 1 nM selenium, 0.1% bovine serum albumin (purified, fatty acid free; fraction V), 4.5 mM nicotinamide, 0.1 nM zinc sulfate heptahydrate, 5 μg/ml transferrin/Fe, 5 μg/ml insulin, 10 μg/ml high density lipoprotein, and a mixture of purified free fatty acids. Its preparation is given in detail in a review on methods. Wauthier, E. et al. Hepatic stem cells and hepatoblasts: identification, isolation and ex vivo maintenance Methods for Cell Biology (Methods for Stem Cells) 86, 137-225 (2008). When used to establish the bioengineered liver, Kubota's Medium was supplemented temporarily with 10% FBS to overcome the enzymes used in preparing a liver cell suspension and then was switched to a serum-free, hormonally defined medium tailored for optimal differentiation of both the parenchymal and non-parenchymal cells and referred to as BIO-LIV-HDM.

Differentiation Medium to Generate Human Liver Tissue (BIO-LIV-HDM).

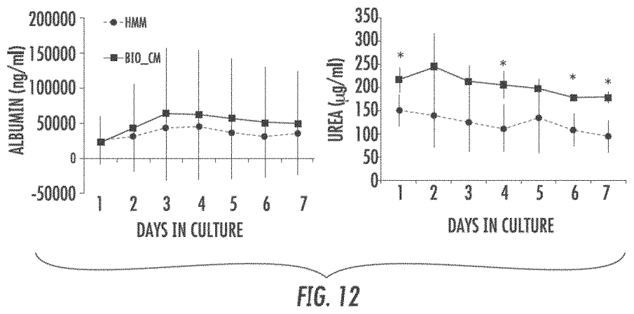

Cells were cultured for 14 days in the bioreactor, following the initial 36 hours of being cultured in seeding medium, in an HDM containing Kubota's Medium supplemented with dexamethasone (0.04 mg/L), prolactin (10 IU/L), glucagon (1 mg/L), nicotinomide (10 mM), Tri-iodothyronine (T3, 67 ng/L), epidermal growth factor (EGF, 20 ng/ml), high-density lipoprotein (HDL, 10 mg/L), hepatocyte growth factor (HGF, 20 ng/ml), human growth hormone (hGH, 3.33 ng/ml), vascular endothelial growth factor (VEG-F, 20 ng/ml), insulin-like growth factor (IGF, 20 ng/ml), cyclic adenosine monophosphate (2.45 mg/L), basic fibroblast growth factor (bFGF, 20 ng/ml), galactose (0.16 grams), angiopoientin (0.2 mg/ml), a mixture of free fatty acids, L-glutamine and antibiotics. This HDM was started 3 days post seeding and then replaced every 2 days afterwards. All reagents were obtained from R&D Systems. The BIO-LIV-HDM proved more successful than the traditional hepatocyte maintenance medium (HMM) in both albumin production and urea secretion in response to the addition of 2 mM ammonia (FIG. 12). However, albumin results were not significantly different due to one human donor sample expressing extremely high levels of albumin compared to the other 2 donors, resulting in a high standard of deviation. Statistical significance will be clarified in the future with additional preparations of adult liver donors, minimizing the standard deviation reported here. All three donors performed comparably in urea secretion. The samples in BIO-LIV-HDM were significantly higher on days 1, 4, 6 and 7 (p<0.05).

### Example 4—Generation of Bioengineered Liver Tissue

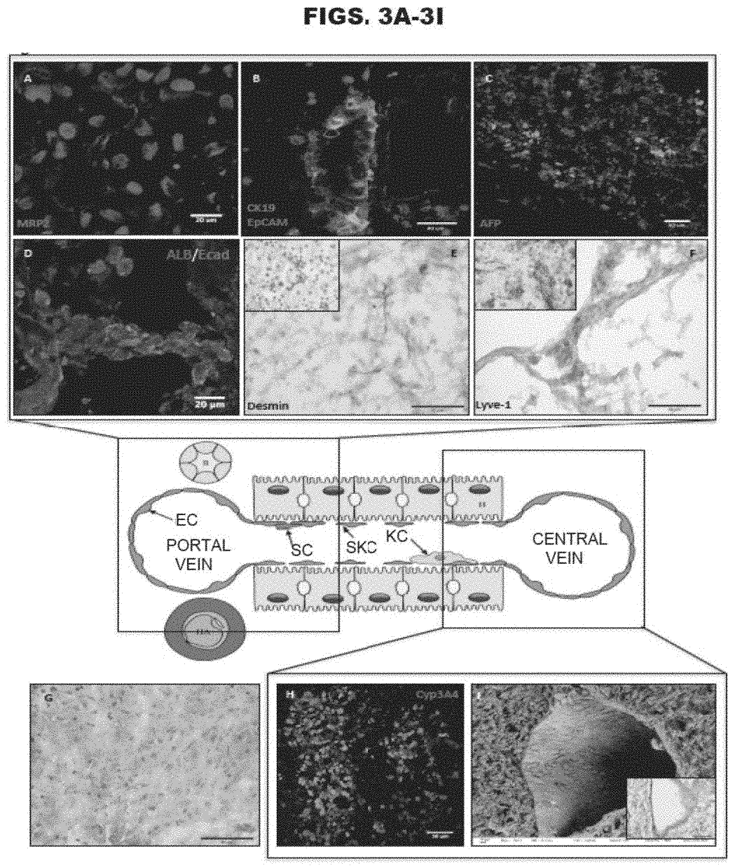

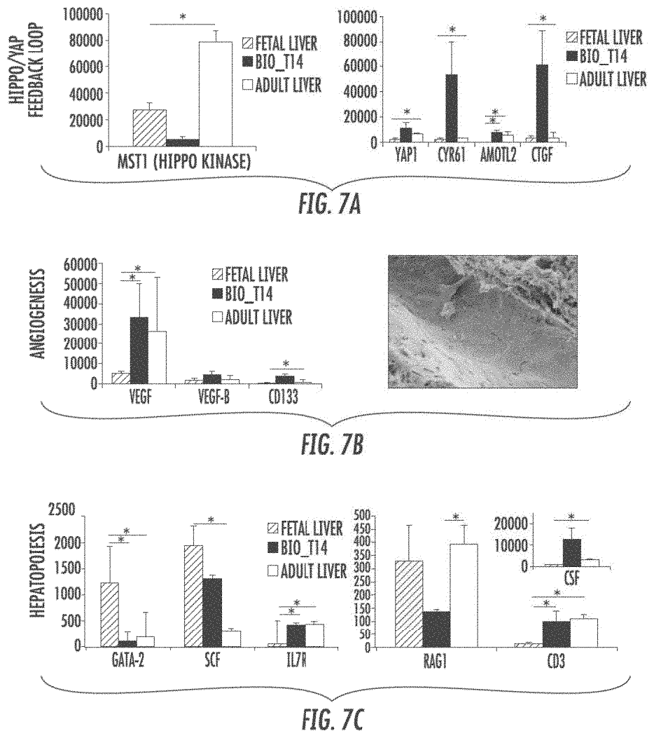

Human hepatic stem/progenitor cells were isolated and stored for 4 hours at 4° C. and in Kubota's medium until seeding. These cells were introduced by perfusion through the matrix remnants of the portal vein via a peristaltic pump and seeded in Kubota's Medium supplemented with 10% FBS (seeding medium). Approximately 90×106 total cells were perfused into a scaffold in 20 min intervals. During each interval, 30×106 cells were perfused at 15 ml/min for 10 min, followed by 10 min of rest (0 ml/min). This was repeated 3 times. Once all of the cells were introduced into a matrix scaffold, the flow rate was lowered to 1.3 ml/min and the scaffolds were perfused with the seeding medium for 36 hrs. Following seeding, the seeding medium was collected, and any cells remaining in the medium were counted with a hemocytometer. The medium was then changed to differentiation medium (BIO-LIV-HDM) that was replaced every 2 days thereafter. The reseeded matrix scaffolds were cultured in the bioreactors for up to 14 days. After 14 days, lobes of the reseeded matrix scaffold were either frozen for histology and immunohistochemistry, fixed for scanning electron microscopy (SEM) and transmission electron microscopy (TEM), or flash-frozen for RNA sequencing (t=14 days). Analyses of these bioreactors are presented as Bio_FL724, Bio_FL728, or Bio_FL732, representing bioreactors seeded with those respective cells. After 36 hrs of seeding, ˜99% of cells had attached to the matrix, evidenced by the lack of cells found in the seeding medium collected and counted by a hemocytometer (data not shown). Upon staining with hematoxylin and eosin (H&E), large numbers of cells were found around the vessels and throughout the parenchyma (data not shown). SEM imaging taken after 14 days in culture revealed endothelial cells lining the vasculature (FIG. 3i and FIG. 7b).

Histology.

(FIG. 3) shows location and expression of proteins identified by immunocytochemistry and immunofluorescence. The expression of mature markers indicates differentiation and re-organization of the fetal liver cells following 14 days in culture. In zone 1, the periportal region, the cells expressed EpCAM and CK19, biomarkers co-expressed in hepatic stem cells and hepatoblasts, and found surrounding the bile ducts. This zone also contains cells that expressed AFP, a biomarker of hepatoblasts. Hepatic cords that had begun to develop are shown in this figure, as well as expression of E-cadherin, a marker of hepatic cell polarity, localized at sites where hepatocytes form cell-cell connections. A marker of biliary transport, MRP2, is identified on the luminal side of hepatic cells, helping to identify cell polarity. It appears that these cells surround a bile duct, indicative of potential biliary functions such as the secretion of bile. Glycogen storage, identified by Periodic Acid-Shiff (PAS) staining, was also evident in cells within the parenchyma. Glycogen can be found in hepatocytes throughout the acinus but those in the periportal region contained the highest levels of glycogen storage. Following along the zonal gradient, cells were found in the parenchyma that expressed markers representative of the peri-central zone (zone 3) such as Cyp3A4, and albumin (found in all zones). In general, the majority of the cells acquired a differentiated state consistent with cells normally found in the periportal region and mature cells found in the mid-acinar and pericentral region.

In addition to cells of the hepatic parenchymal cell lineages, stellate cells, identified by their expression of desmin, and sinusoidal endothelial cells, lyve-1+ cells, were found localized to locations in the scaffold corresponding to those in vivo. Stellate cells typically co-localize with their epithelial partners, requisite for paracrine signaling involved in mitosis and specialized cell functions. The shape of these cells in the histology pictures was slim, because the cells were squeezed in the process of wrapping around cells (positive control pictures are shown for reference). Cells expressing alpha-smooth muscle actin (αSMA) were found around vessel structures. The αSMA positive cells were possibly pericytes, which can be activated to proliferate along with endothelial cells, CD31+ cells, found lining the blood vessels. They were evident by both immunohistochemistry and SEM (FIG. 3i). Proliferation was evident by Ki67 staining (data not shown) and mostly found in cells located around blood vessels. Larger cells did not stain positive for Ki67 and, therefore, are assumed to be in a non-proliferative, fully mature state.

RNA Sequencing.

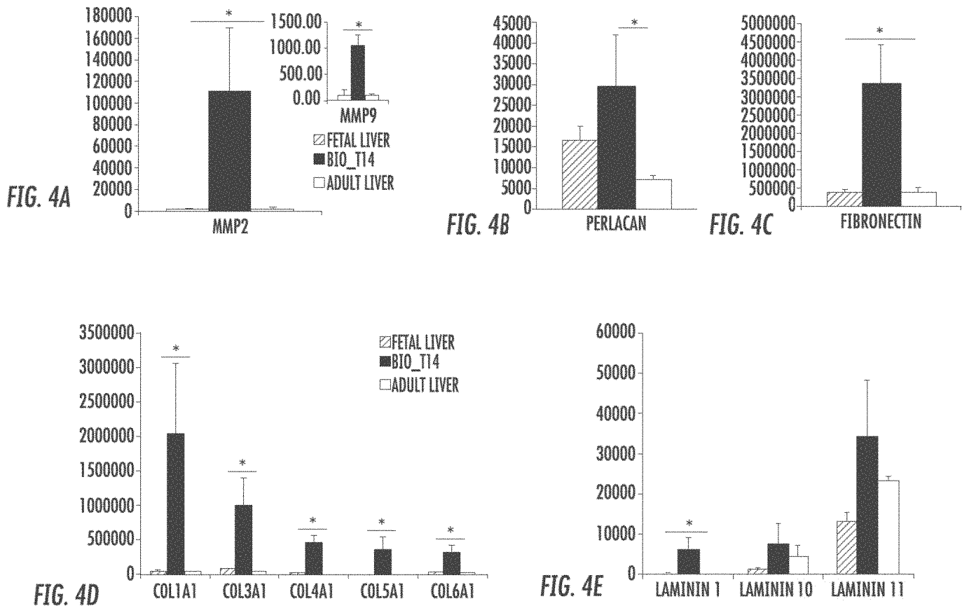

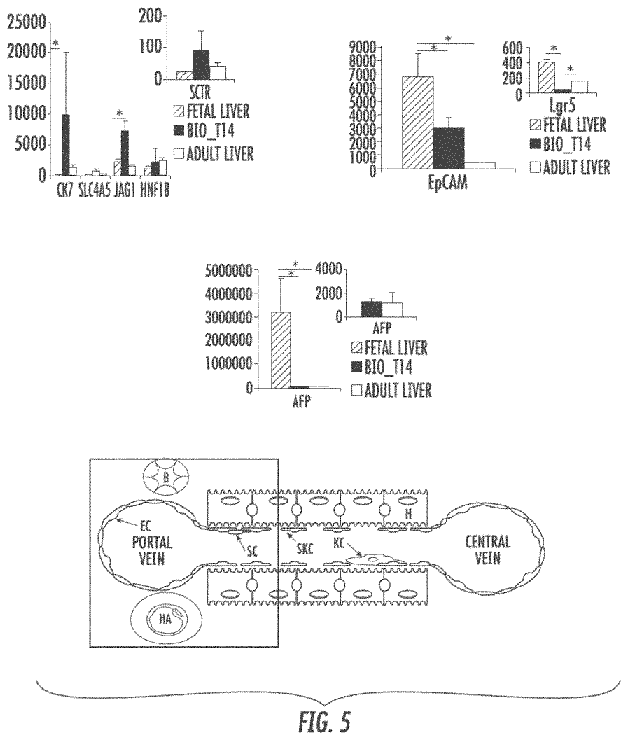

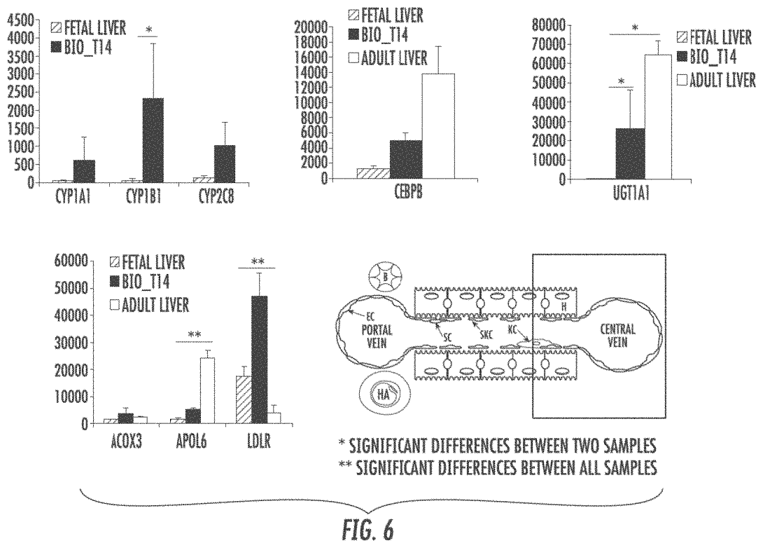

We performed paired-end high-throughput RNA sequencing on the samples from the three different bioreactors obtaining an average of ˜200 million paired-end reads per sample, of which an average of ˜87% mapped uniquely to the human genome. A number of facets of functionality and stages of differentiation have been identified by analyzing the RNA sequencing data. Firstly, it is apparent that cells within the bioreactors were remodeling the matrix, identifiable by the increased expression of MMP-2 and MMP-9 (matrix degradation enzymes, FIG. 4a) and the increased expression of collagens, laminins, fibronectin, and perlecan (FIG. 4b-e). The mRNA expression levels for these genes were all significantly higher in the bioreactors compared to those in both fetal and adult liver samples (p<0.05), with the exception of perlecan, for which the bioreactor was only significantly greater than adult liver cells, and laminin 10 and 11, where there was no significant difference between samples. There were also indications that the fetal liver-derived stem/progenitor cells in the bioreactor had differentiated to represent all maturational parenchymal cell lineage stages, evident by decreased expression of fetal genes and up-regulation of more mature genes (FIGS. 5 and 6). Fetal genes such as LGR5 and EpCAM, known markers for hepatic stem cells, were significantly lower in expression levels in the bioreactor samples compared to the fetal cells, with a 3.4 and 1.2 fold change respectively (FIG. 5). Similarly, the gene most known to identify hepatoblasts, AFP, was more than 11 fold greater in fetal tissue compared to the bioreactor samples and adult liver tissue; there was no significant difference between the levels in the bioreactors and adult liver tissue.

In contrast, the levels of gene expression for mature hepatic markers rose steadily within less than a week in the bioreactor samples compared to fetal tissue, indicating maturational development of the hepatic parenchymal cell lineages. In zone 1, mature biliary markers CK7, SLC4A2, JAG1, HNF1B and SCTR (FIG. 6) were all up-regulated compared to fetal and adult liver samples. Most significantly increased compared to fetal liver cells were CK7, JAG1 and SCTR, which were greater than 98, 1.75 and 1.85 fold higher respectively. Expression levels of zone 3 markers of metabolic function that were up-regulated in the bioreactor samples included mature forms of P450 genes (CYP1A1, CYP-1B1 and CYP-2C8); all genes had at least a >3 fold increase relative to fetal cells; UDP-glucoronyl transferase UTG1A1, which was increased by ˜10 fold compared to fetal cells; and genes involved in lipid and cholesterol metabolism (ACOX3, APOL6, LDLR), although only significantly higher in LDLR. This maturity was further suggested by a greater than 4-fold decrease in expression of CYP3A7, the fetal form of P450, in the bioreactor tissue compared to the fetal liver samples (data not shown). Another marker for mature hepatocytes, C/EBP, was also increased in the bioreactor, although not significantly, and no change was seen in HNF4a expression compared to fetal liver.

The gene expression levels measured in the bioreactors, while primarily at levels suggesting maturation beyond that in fetal liver, were in most cases still distinct from those in the adult tissue. This suggests that additional time in culture or modified culture conditions (e.g. further reduction in the use of serum, greater regulation of the oxygenation) are required for further maturation. With that in mind, the gene expression levels of Yap, the related targeting genes, and Hippo all indicate that the regenerative process was active. The gene expression level of MST1, a Hippo kinase, was significantly lower in the bioreactor compared to fetal and adult liver; in parallel, the Yap signaling genes were all significantly increased in the bioreactor compared to fetal and adult liver (FIG. 7a). Gene expression of angiogenic markers indicated that the bioreactor tissues were undergoing angiogenesis and vasculogenesis (FIG. 7b). Expression levels of VEGF, VEGF-B and CD133 were all increased in the bioreactor samples compared to fetal liver cells, showing especially significant differences in VEGF and CD133 (p<0.05).

Based on RNA sequencing data, there are suggestions of hematopoietic differentiation (FIG. 7c). Markers of earlier hematopoietic stem cells (Gata-2, SCF and IL-7R) were down-regulated in bioreactor samples, transitioning from levels found in fetal tissues to levels matching those in the adult livers. Simultaneously, genetic profiles of mature hematopoietic cells in the lymphoid and myeloid lineages also differ between the fetal liver, bioreactor and adult liver. Bioreactor samples have gene expression levels of CD3 similar to those found in adult liver; Rag1 expression rising (both genes (Rag1 and CD3 are associated with T cells), and CSF expression (expressed by myeloid cells) isare significantly higher compared to both fetal and adult livers. These markers are indicative of possible hematopoiesis, but more extensive analyses are required to allow for accurate interpretations.

Cell Viability.

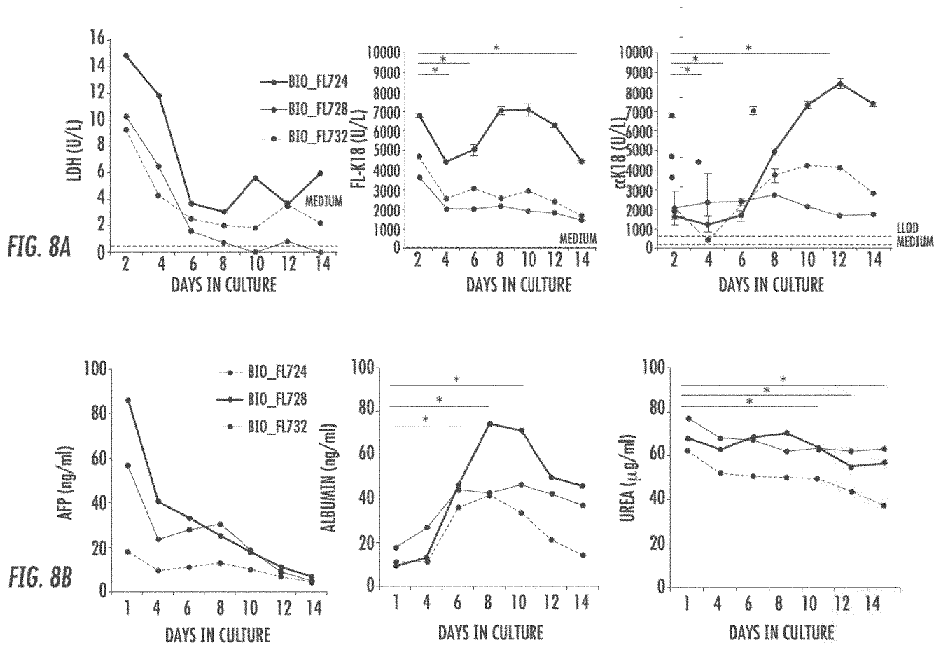

ALT and AST, aminotransferases enzymes used to evaluate liver cell health, were assessed on days 2, 4, 6, 8, 10, 12 and 14. At no time during the course of this experiment did levels of ALT exceed the lower limit of detection (data not shown). Thus, it was determined that it is not a sensitive biomarker for this ex vivo model system. Bio_FL724 was the only bioreactor that had measurable levels of AST over the lower limit of detection (4 U/L) throughout the entire time in culture (data not shown). LDH levels (FIG. 8a) for each bioreactor were initially high but decreased over time. Following the first day in culture, however, measurements for LDH at each time point were significantly lower than the initial measurement (p<0.05). The interpretation of this data is that in the initial few days, there were cells with greater turnover due to stress from the isolation procedure and/or seeding process. After this recovery period, the cells generated phenotypic traits suggesting rapid liver organogenesis.

Full length K18 (FL-K18) levels in the medium (therefore, secreted or released from cells) is specific for necrosis; values were above baseline (25.3 U/L) in all bioreactors. The trend in FL-K18 levels was similar in all three bioreactors. Levels were significantly high on day 2 (FIG. 8a) and are assumed to be due to cellular stress or damage from the isolation procedures. Following day 2 there was a significant decrease in levels, with days 4 and 6 days significantly less than the initial reading at day 2 (p<0.05). The initial fall in FL-K18, suggestive of less necrotic cells in culture, could be in response to complete cell death and, specifically in the case of Bio_FL728 and Bio_FL732, the remaining cells indicate that there is a selection of healthier cells. However, there was an increase in FL-K18 during days 8-12 and then levels fell again.

The levels of ccK18 (FIG. 8a) that were detected follow a similar trend as FL-K18 levels, in that levels begin to rise around day 6, peak around day 8 and then decrease. Although this data might suggest high apoptotic conditions, there were no significant differences in levels throughout the entire time in culture, suggesting that there were no significant increases in apoptosis over time.

Overall, the data describing cell viability and health suggested that the cells experience a transient period of 2-3 days when cells damaged in the isolation and seeding process are eliminated, followed by stabilization of the remaining cells and then their differentiation.

The rise in FL-K18 and ccK18 also corresponded to increase secretion of albumin by cells in all three bioreactors. It is hypothesized that this increase resulted from terminally differentiated polyploid hepatocytes undergoing apoptosis as part of a normal cell cycle process. Following this peak in apoptosis, ccK18 levels immediately fell, which suggests that precursor cells are undergoing maturation to replace the lost pericentral hepatocytes.

AFP (FIG. 8b).

Each bioreactor demonstrated distinct starting levels of AFP on day 2, the first day of sample collections, corresponding with the differences in gene expression between fetal liver cells' at t=0. Regardless of the initial values of AFP, there was a dramatic drop in production over time.

Albumin (FIG. 8b).

The albumin production levels in all three bioreactors were initially low but rose steadily over time, with significantly higher levels between days 6-10 (p<0.05). The actual amount of albumin produced by the individual bioreactors differed, but the general trend of an increase in production was consistent among all bioreactors. The level peaked by day 8 and decreased by day 10. It is hypothesized that the rise and fall corresponded to cells differentiating to late lineage stage, atocytes indicated by the high production of albumin. They subsequently underwent apoptosis, which led to a decrease in albumin production as precursor cells continue the regenerative process. This interpretation of the data is also supported by the ccK18 levels measured at the respective time points.

Urea (FIG. 8b).

Unlike the production of AFP and albumin, the levels of urea did not dramatically change over the course of 14 days. All three bioreactors had the largest amount of urea secretion on day 2 and decreased slightly thereafter. By day 10, the levels of urea were significantly lower than the initial values on day 2 (p<0.05), although overall it appeared that secretion remained steady over time.

Cell Metabolomics

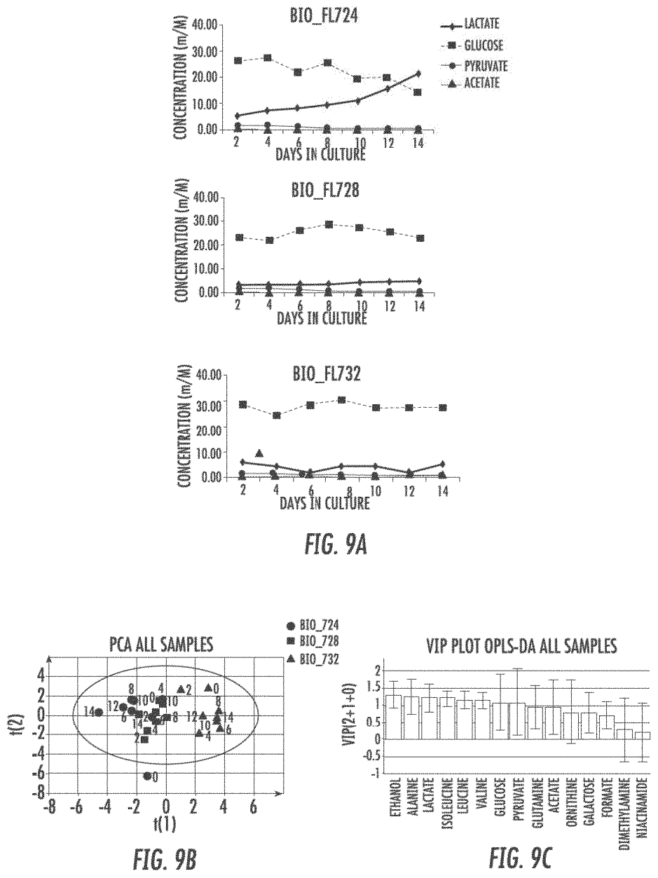

Functionality of cells was assessed by metabolic activity and measured by nuclear magnetic resonance (NMR) spectroscopy (FIG. 9). Principle component analysis (PCA, FIG. 9b) was performed indicating that two of the bioreactors, Bio_FL724 and Bio_FL732, responded more similarly in culture compared to the third, Bio_FL732. In all bioreactors the cells consumed and metabolized glucose, glutamine, pyruvate and acetate that were provided in the medium and converted them to the production of lactate (FIG. 9a). These actions show conclusively that the cells were undergoing glycolysis and entering the Kreb cycle. Bio_FL724 was immediately active by day 2, and Bio_FL728 had similar trends by day 6. The third bioreactor, Bio_FL732 became metabolically active by day 8, although at much lower levels than the other two bioreactors. This suggests that there was a lag-time in which the two bioreactors, Bio_FL728 and Bio_FL732, needed to recover from possible stress from the seeding process or that the cells, upon isolation, were not as healthy as Bio_FL724 and required more time to become metabolically active. The VIP plot (FIG. 9c) shows the metabolites that contribute to the separation. VIP>=1.0 is considered important.

Transmission Electron Microscopy (TEM)

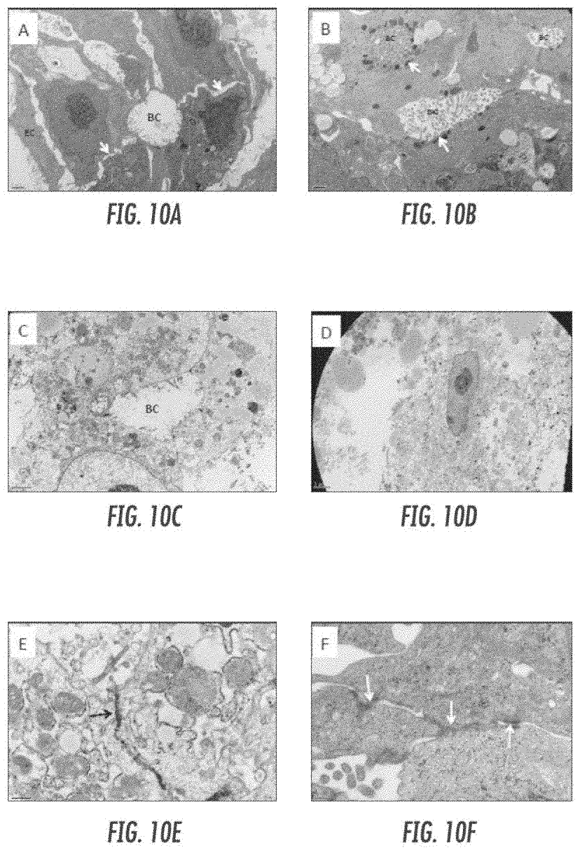

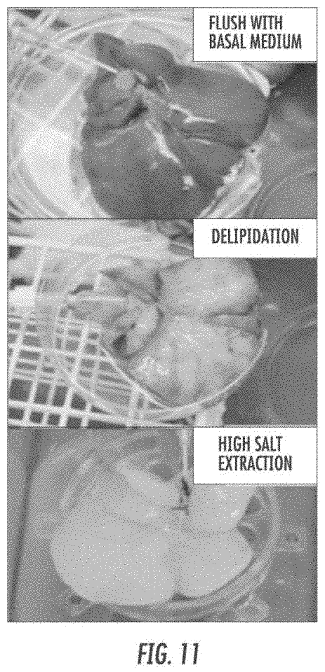

The organization of the cells in their respective bioreactors was further evaluated by TEM. In order to be functional, epithelial cells must form cell-cell connections that are instrumental in cell polarity, cell signaling with neighboring cells, and interactions with the matrix. Components of junctional complexes (FIG. 10e,f) were visualized by TEM imaging as hepatocytes came together to form sheets, or plates, with bile canaliculi (FIG. 10a-c) between them, an essential arrangement for transporting secreted bile. Sinusoidal spaces were observed between these hepatocyte-like cells (FIG. 10a) and possible secretory vesicles were seen around the bile canaliculi spaces (FIG. 10b). In addition to hepatic cells, there were several cells with physical characteristics suggestive of endothelial cells, stellate (Ito) cells, and stem cells in the process of differentiation, identified by TEM (data not shown). The seeding of cells was not homogeneous throughout the entire biomatrix scaffold resulting in sites with varying stages of cells within the organogenesis process findings indicated in the TEM images. There were lipid droplets seen in the images not associated with cells (data not shown), which can be an indication of cell breakup either during preparation of the sample for imaging or could have occurred during the aging process of the cells in culture, similar to that represented in the necrosis and apoptosis data.

### Experiment 5: Characterization of Collagens in Scaffold

The tissue is rinsed to minimize the amount of blood and interstitial fluid. Most fibrillar collagens cannot be extracted with the typical initial rinse that folks use: phosphate buffered saline (PBS). However, uncross-linked collagens and associated matrix components including procollagens, collagen monomers (before fibrils are formed) and non-fibrillar collagen types (e.g. type IV, type VI), can be extracted with PBS. Thus, the initial rinse is performed with a basal medium (a mix of amino acids, nutrients, lipids, vitamins, trace elements, etc). and at an ionic strength that will not cause the collagens to go into solution.

The delipidation steps used by others and the long (sometimes hours or even days (!!) to which the tissue is subjected to delipidation. SDS binds to the matrix very tightly and makes it toxic. Triton-X and other such harsh detergents solubilize various matrix components. One procedure uses SDS followed by Triton-X, a procedure that results in “very clean” scaffolds but, in fact, they look “clean” because so much has been lost. Thus, a low concentration of a bile salt, sodium deoxycholate, and in combination with phospholipase that results in rapid and very gentle delipidation. A dilapidation is conducted in 20-30 minutes.

Extraction is carried out using low ionic strength buffers (ones under 1 M NaCl) result in significant loss of uncross-linked collagens; those at 1 M NaCl preserve some collagens (mostly type I collagen) but not all (not network collagens). Thus, the present method does not lose any of the collagens (fibrillary or network; cross-linked or uncrosslinked) and so preserve everything bound to them. In contrast, methods using distilled water may lose all but the highly cross-linked collagens as well as the components bound thereto, which are solubilized in the water.

Nucleic acids are removed according to methods standard in the art.

The distinctions obtained by isolating bomatrix scaffolds by are characterized by collecting the supernatants, dialyze them, lyophilize them, and measure collagen content in them by amino acid, cross-link, Western blot, and growth factor analysis. This will determine the collagens preserved by this method. Parallel extractions are performed using a) with PBS; b) with low ionic strength buffers; c) after their various delipidation methods; d) with distilled water. The supernatant from each of these steps is collected and subjected to amino acid analysis to assess if collagens are lost and the extent of loss. Where collagens amounts are determined to be substantial, the collagens in the supernatants are treated with [3H]—NaBH4, hydrolyze and subject it to cross-link analysis. In addition, Western blot analysis with antibodies is run to identify the extent of cross-linking and the types of collagens present. Further, growth factor analysis will be performed to characterize the resulting scaffolds.

### EXEMPLARY EMBODIMENTS

Non-limiting exemplary embodiments are provided herein below:

[1] A container for the generation of bioengineered tissue, where the generation comprises introducing epithelial and mesenchymal cells into or onto a biomatrix scaffold, wherein the biomatrix scaffold comprises collagens.
[2] The container of [1], in which epithelial and mesenchymal cells are maturational lineage partners.
[3] The container of [1] or [2], in which epithelial and mesenchymal cells are in a seeding medium, and the seeding medium is replaced with a differentiation medium after an initial incubation period.
[4] The container of [3], where in the differentiation medium comprises:
a. A basal medium,
b. Lipids, insulin, transferrin, antioxidants,
c. Copper,
d. Calcium,
e. One or more signals for the propagation or maintenance of epithelial cells, and/or
f. One or more signals for the propagation or maintenance of mesenchymal cells.
[5] The container of [3] or [4] in which the seeding medium is serum-free or is supplemented with between about 2% to 10% fetal serum, optionally over the duration of a few hours.
[6] The container of [3] to [5], where in the seeding medium comprises:
a. A basal medium
b. Lipids
c. Insulin
d. Transferrin
e. Antioxidants.
[7] The container of any one of [3] to [6] in which the epithelial and mesenchymal cells in the seeding medium is incubated at 4° C. in the seeding medium for 4 to 6 hours prior to introduction into the biomatrix scaffolds
[8] The container of any one of [1] to [7], in which the biomatrix scaffold is three-dimensional
[9] The container of any one of [1] to [8], in which the collagens in the biomatrix scaffold comprise (i) nascent collagens, (ii) aggregated but not cross-linked collagen molecules, (iii) cross-linked collagens.
[10] The container of any one of [1] to [9] in which the epithelial and mesenchymal cells in the seeding medium are introduced in multiple intervals, each interval followed by a period of rest.
[11] The container of [10] in which the interval is about 10 minutes and the period of rest is about 10 minutes.
[12] The container of [10] or [11] in which the seeding density is up to about 12 million cells per gram of wet weight of the biomatrix scaffolds and introduced during one or more intervals.
[13] The container of any one of [10] to [12] in which the epithelial and mesenchymal or non-parenchymal cells in the seeding medium are introduced at a rate of ˜15 ml/min for one or more intervals.
[14] The container of any one of [10] to [13], in which the epithelial and mesenchymal cells in the seeding medium are introduced in 10 minute intervals, each followed by a 10 minute period of rest.
[15] The container of any one of [10] to [14] in which the epithelial and mesenchymal cells in the seeding medium are is introduced at a rate of 1.3 ml/min after three intervals.
[16] The container of any one of [1] to [15] in which the epithelial and mesenchymal cells comprise cells isolated from a fetal or neonatal organ.
[17] The container any one of [1] to [15] in which the epithelial and mesenchymal cells comprise cells isolated from an adult or child donor
[18] The container of any one of [1] to [17] in which the epithelial and mesenchymal cells comprise:
a. epithelial cells comprising one or more of stem cells, committed progenitors, diploid adult cells, polyploid adult cells, and/or terminally differentiated cells
and/or
b. mesenchymal cells comprising one or more of angioblasts, precursors to endothelia, mature endothelia, precursors to stellate cells, mature stellate cells, precursors to stroma, mature stroma, smooth muscle cells, precursors to hematopoietic cells, and/or mature hematopoietic cells.
[19] The container of any one of [1] to [18] in which the epithelial and mesenchymal cells comprise:
a. epithelial cells comprising one or more of biliary tree stem cells, gall bladder-derived stem cells, hepatic stem cells, hepatoblasts, committed hepatocytic and biliary progenitors, axin2+ progenitors (such as axin2+ hepatic progenitors), mature parenchymal cells (such as hepatocytes, cholangiocytes), pancreatic stem cells, and pancreatic committed progenitors, islet cells, and/or acinar cells, and/or
b. mesenchymal cells comprising one or more of angioblasts, stellate cell precursors, stellate cells, mesenchymal stem cells, pericytes, smooth muscle cells, stromal cells, endothelial cell precursors, endothelial cells, hematopoetic cell precursors, and/or hematopoetic cells.
[20] The container of any one of [1] to [19] in which the epithelial cells comprises one or more of stem cells and their descendants from the biliary tree, liver, pancreas, hepato-pancreatic common duct, and/or gall bladder and/or mesenchymal cells comprising one or more of angioblasts, precursors to endothelia and stellate cells, mesenchymal stem cells, stellate cells, stroma, smooth muscle cells, endothelia, bone marrow-derived stem cells, hematopoetic cell precursors, and/or hematopoetic cells.
[21] The container any one of [1] to [20] in which the epithelial and mesenchymal cells consists of about 80% epithelial and 20% mesenchymal respectively
[22] The container of any one of [1] to [21] in which the epithelial and mesenchymal cells comprise at least 50% stem cells and/or precursor cells.
[23] The container of any one of [1] to [22], wherein the epithelial and mesenchymal cells do not comprise any terminally differentiated hepatocytes and/or pancreatic cells.
[24] The container of any one of [1] to [23] in which the biomatrix scaffold comprises one or more collagen associated matrix components comprising one or more of laminins, nidogen, elastins, proteoglycans, hyaluronans, non-sulfated glycosaminoglycans, sulfated glycosaminoglycans, growth factors and/or cytokines associated with the matrix components.
[25] The container of any one of [1] to [24] in which the biomatrix scaffold comprises greater than 20-50% of matrix-bound signaling molecules found in vivo.
[26] The container of any one of [1] to [25] in which the biomatrix scaffold comprises a matrix remnant of the vascular tree of the tissue and/or wherein the matrix remnant provides vascular support of the cells in the bioengineered tissue
[27] A three-dimensional scaffold comprising extracellular matrix, which in turn comprises (i) native collagens found in an organ and/or (ii) matrix remnants of a vascular tree found in an organ
[28] A three-dimensional micro-organ generated in the container of any one of [1] to [26].
[29] A bioengineered tissue comprising zonation-dependent phenotypic traits characteristic of native liver, said phenotypic traits including (a) periportal region having traits of stem/progenitors, diploid adult cells and/or associated mesenchymal precursor cells, (b) a mid-acinar region having cells with traits of sinusoidal plates of mature parenchymal cells and mesenchymal cells, and/or (c) a pericentral region having traits of terminally differentiated epithelial and, apoptotic cells associated with fenestrated endothelia and/or axin2+ hepatic progenitors that are connected to endothelia of the central vein.
[30] The bioengineered tissue of [29] in which the phenotypic traits further include traits associated with diploid epithelial cells and/or mesenchymal cells of the periportal zone
[31] The bioengineered tissue of [29] or [30] in which the phenotypic traits further include traits of mature epithelial cells and/or mesenchymal cells found in the mid-acinar region of native liver.
[32] The bioengineered tissue of any one of [29] to [31] in which the phenotypic traits further include traits of epithelial or parenchymal and/or mesenchymal cells of the pericentral zone.
[33] The bioengineered tissue of any one of [29] to [32] further comprising: (i) polyploid hepatocytes associated with fenestrated endothelial cells, and/or (ii) diploid hepatic progenitors (such as axin2+ cells) connected to endothelia of a central vein
[34] The bioengineered tissue of any one of [29] to [33] in which the periportal region is enriched in traits of the stem/progenitor cell niches that comprise hepatic stem cells, hepatoblasts, committed progenitors, and/or diploid adult hepatocytes.
[35] The bioengineered tissue of any one of [29] to [34] in which the epithelial and mesenchymal cells further comprise epithelial cells comprising precursors and/or mature forms of hepatocytes and/or cholangiocytes.
[36] The bioengineered tissue of any one of [29] to [35] in which the epithelial and mesenchymal cells further comprise mesenchymal cells comprising precursors and/or mature forms of stellate cells, pericytes, smooth muscle cells, stroma, endothelia and/or hematopoietic cells
[37] A three-dimensional micro-organ comprised of the bioengineered tissue of any one of [29] to [36].
[38] The three-dimensional micro-organ of [37] generated in the container of any one of [1] to [26].
[39] A kit for culturing the micro-organ in the container of any one of [1] to [26] with accompanying instructions.
[40] A method of evaluating a treatment for an organ comprising administering the treatment to a bioengineered tissue or three-dimensional micro-organ of any one of [29] to [38].
[41] A differentiation medium for both epithelial and mesenchymal cells comprising
a. A basal medium containing lipids, insulin, transferrin, antioxidants,
b. Copper,
c. Calcium,
d. One or more signals for the propagation and/or maintenance of epithelial cells, and/or
e. One or more signals for the propagation and/or maintenance of mesenchymal cells.
[42] The differentiation medium of [41] in which the basal medium is Kubota's Medium.
[43] The differentiation medium of [41] or [42] further comprising one or more lipid binding proteins.
[44] The differentiation medium of [43] in which the one or more lipid binding proteins is high-density lipoprotein (HDL).
[45] The differentiation medium of any one of [41] to [44] further comprising one or more purified fatty acids.
[46] The differentiation medium of [45] in which the one or more purified fatty acids comprises palmitic acid, palmitoleic acid, stearic acid, oleic acid, linoleic acid, and/or linolenic acid.
[47] The differentiation medium of any one of [41] to [46] further comprising one or more sugars.
[48] The differentiation medium of any one of [47] in which the one or more sugars comprises galactose, glucose, and/or fructose.
[49] The differentiation medium of any one of [41] to [48] further comprising one or more glucocorticoids.
[50] The differentiation medium of [49] in which the one or more glucocorticoids comprises dexamethasone and/or hydrocortisone
[51] A bioengineered tissue comprising zonation-dependent phenotypic traits characteristic of native pancreas and/or that includes zonation associated with pancreatic cells in the head of the pancreas and/or those associated with pancreatic cells in the tail of the pancreas.

1.-27. (canceled)

28. A bioengineered tissue generated by introducing epithelial and mesenchymal cells into or onto a biomatrix scaffold, wherein the biomatrix scaffold comprises collagens, and wherein the epithelial and mesenchymal cells are maturational lineage partners.

29. The bioengineered tissue of claim 28, in which the epithelial and mesenchymal cells are in a seeding medium, and the seeding medium is replaced with a differentiation medium after an initial incubation period.

30. The bioengineered tissue of claim 29, in which the differentiation medium comprises:
a) a basal medium;
b) lipids, insulin, transferrin, antioxidants;
c) copper;
d) calcium;
e) one or more signaling molecules for the propagation or maintenance of epithelial cells; and/or
f) one or more signaling molecules for the propagation or maintenance of mesenchymal cells.

31. The bioengineered tissue of claim 29, in which the seeding medium is serum-free or is supplemented with between about 2% to 10% fetal serum.

32. The bioengineered tissue of claim 29, in which the seeding medium comprises:
a) a basal medium;
b) lipids;
c) insulin;
d) transferrin; and/or
e) antioxidants.

33. The bioengineered tissue of claim 29, in which the epithelial and mesenchymal cells in the seeding medium is incubated at 4° C. in the seeding medium for 4 to 6 hours prior to introduction into the biomatrix scaffold.

34. The bioengineered tissue of claim 28, in which the biomatrix scaffold is three-dimensional.

35. The bioengineered tissue of claim 28, in which the collagens in the biomatrix scaffold comprises (i) nascent collagens, (ii) aggregated but not cross-linked collagen molecules, and/or (iii) cross-linked collagens.

36. The bioengineered tissue of claim 29, in which the epithelial and mesenchymal cells in the seeding medium are introduced in multiple intervals, and in which an interval of introducing the epithelial and mesenchymal cells is followed by a period of rest.

37. The bioengineered tissue of claim 36, in which the interval is about 10 minutes and the period of rest is about 10 minutes.

38. The bioengineered tissue of claim 36, in which the mesenchymal and epithelial cells are introduced with a seeding density of up to about 12 million cells per gram of wet weight of the biomatrix scaffold.

39. The bioengineered tissue of claim 36, in which the epithelial and mesenchymal in the seeding medium are introduced at a rate of ˜15 ml/min.

40. The bioengineered tissue of claim 36, in which the epithelial and mesenchymal cells in the seeding medium are introduced in 10 minute intervals, each followed by a 10 minute period of rest.

41. The bioengineered tissue of claim 36, in which the epithelial and mesenchymal cells in the seeding medium are introduced at a rate of 1.3 ml/min after three intervals.

42. The bioengineered tissue of claim 28, in which the epithelial and mesenchymal cells comprise cells isolated from a fetal or neonatal organ.

43. The bioengineered tissue of claim 28, in which the epithelial and mesenchymal cells comprise cells isolated from an adult or child donor

44. The bioengineered tissue of claim 28, in which
a) the epithelial cells comprise one or more of stem cells, committed progenitors, diploid adult cells, polyploid adult cells, or terminally differentiated cells;
and
b) the mesenchymal cells comprise one or more of angioblasts, precursors to endothelia, mature endothelia, precursors to stellate cells, mature stellate cells, precursors to stroma, mature stroma, smooth muscle cells, precursors to hematopoietic cells, or mature hematopoietic cells.

45. The bioengineered tissue of claim 28, in which
a) the epithelial cells comprise one or more of biliary tree stem cells, gall bladder-derived stem cells, hepatic stem cells, hepatoblasts, committed hepatocytic and biliary progenitors, axin2+ progenitors, mature hepatic parenchymal cells, hepatocytes, cholangiocytes, pancreatic stem cells, and pancreatic committed progenitors, islet cells, or acinar cells; and
b) the mesenchymal cells comprise one or more of angioblasts, stellate cell precursors, stellate cells, mesenchymal stem cells, pericytes, smooth muscle cells, stromal cells, endothelial cell precursors, endothelial cells, hematopoetic cell precursors, or hematopoetic cells.

46. The bioengineered tissue of claim 28, in which a) the epithelial cells comprise one or more of stem cells and their descendants from the biliary tree, liver, pancreas, hepato-pancreatic common duct, or gall bladder; and b) in which the mesenchymal cells comprise one or more of angioblasts, precursors to endothelia and stellate cells, mesenchymal stem cells, stellate cells, stroma, smooth muscle cells, endothelia, bone marrow-derived stem cells, hematopoetic cell precursors, or hematopoetic cells.

47. The bioengineered tissue of claim 28, in which the epithelial and mesenchymal cells consists of about 80% epithelial cells and 20% mesenchymal cells, respectively.

48. The bioengineered tissue of claim 28, in which the epithelial and mesenchymal cells comprise at least 50% stem cells and/or precursor cells.

49. The bioengineered tissue of claim 28, wherein the epithelial and mesenchymal cells do not comprise any terminally differentiated hepatocytes and/or pancreatic cells.

50. The bioengineered tissue of claim 28, in which the biomatrix scaffold comprises one or more collagen associated matrix components comprising one or more of laminins, nidogen, elastins, proteoglycans, hyaluronans, non-sulfated glycosaminoglycans, sulfated glycosaminoglycans, growth factors and/or cytokines associated with the matrix components.

51. The bioengineered tissue of claim 28, in which the biomatrix scaffold comprises from about 20 to about 50% of matrix-bound signaling molecules found in vivo.

52. The bioengineered tissue of claim 28, in which the biomatrix scaffold is derived from a tissue, wherein the tissue comprises a vascular tree, wherein the biomatrix scaffold comprises a matrix remnant of the vascular tree of the tissue, and wherein the matrix remnant provides vascular support of the epithelial and mesenchymal cells in the bioengineered tissue.

53. The bioengineered tissue of claim 28, wherein the bioengineered tissue is a bioengineered liver, wherein the biomatrix scaffold is derived from a liver, wherein the biomatrix scaffold comprises a matrix remnant of a liver vascular tree, and wherein the epithelial cells are hepatic stem/progenitor cells.

54. The bioengineered tissue of claim 53, wherein the bioengineered liver comprises a capacity to secrete albumin or bile, a capacity to metabolize one or more of glucose, glutamine, pyruvate, or acetate; and/or a capacity to metabolize drugs.

55. The bioengineered tissue of claim 54, wherein the capacity to metabolize drugs is handled by a P450 enzyme.

56. The bioengineered tissue of claim 28, wherein the bioengineered tissue comprises a plurality of epithelial cell-cell connections.

57. The bioengineered tissue of claim 56, wherein the plurality of epithelial cell-cell connections comprises a plurality of junctional complexes formed between the epithelial cells as determined by transmission electron microscopy.

58. The bioengineered tissue of claim 56, wherein the bioengineered tissue is a liver, and the plurality of epithelial cell-cell connections forms a sheet of hepatocytes with a bile canaliculi between them.

59. A method of preparing a bioengineered tissue according to claim 28.
